(19) United States
(12) Patent Application Publication
(10) Pub. No.: US 2008/0279869 A1
(43) Pub. Date: Nov. 13, 2008

(54) METHOD FOR REDUCING NEURONAL DEGENERATION BY ADMINISTERING CNS-DERIVED PEPTIDES OR ACTIVATED T CELLS

(75) Inventors: Michal Eisenbach-Schwartz, Rehovot (IL); Ehud Hauben, Hadera (IL)

Correspondence Address: BROWDY AND NEIMARK, P.L.L.C.;624 NINTH STREET, NW, SUITE 300, WASHINGTON, DC, 20001-5303, US

(73) Assignee: Yeda Research and Development Co., Ltd., Rehovot (IL)

(21) Appl. No.: 11/563,630

(22) Filed: Nov. 27, 2006

Related U.S. Application Data
(63) Continuation of application No. 10/466,220, filed on Jan. 5, 2004, now abandoned.
Application No. 10/466,220 is a continuation-in-part of application No. 10/810,653, filed on Mar. 29, 2004.
Application No. 10/810,653 is a continuation of application No. 09/893,348, filed on Jun. 28, 2001, now abandoned.
Application No. 09/893,348 is a continuation-in-part of application No. 09/314,161, filed on May 19, 1999, now abandoned.
Application No. 09/314,161 is a continuation-in-part of application No. PCT/US98/14715, filed on Jul. 21, 1998.
Application No. PCT/US98/14715 is a continuation-in-part of application No. 09/218,277, filed on Dec. 22, 1998, now abandoned.

(30) Foreign Application Priority Data
May 19, 1998 (IL) 124500
Jan. 14, 2001 (IL) 140888

(51) Int. Cl. A61K 39/00; A61P 25/28; A61P 9/10; A61P 25/02

(52) U.S. Cl. 424/184.1

(57) ABSTRACT

Compositions are provided for promoting nerve regeneration or reducing or inhibiting degeneration in the CNS or PNS to ameliorate the effects of injury or disease. The composition includes an active ingredient selected from:(a) a peptide obtained by modification of a self-peptide derived from a CNS-specific antigen, which modification consists in the replacement of one or more amino acid residues of the self-peptide by different amino acid residues, such modified CNS peptide still being capable of recognizing the T-cell receptor recognized by the self-peptide but with less affinity; (b) a nucleotide sequence encoding such a peptide; (c) T cells activated by such peptide; and (d) any combination of (a)-(c). The peptide is preferably obtained by modification of the self-peptide p87-99 of MBP, more preferably, by replacing lysine 91 with glycine (G91) or alanine (A91) or by replacing proline 96 with alanine (A96).

### FIELD OF THE INVENTION

The present invention relates to pharmaceutical compositions comprising modified central nervous system (CNS)-derived peptides and methods for the promotion of nerve regeneration in the CNS and the peripheral nervous system (PNS). The present invention also relate to the use of these peptides for vaccination or for activation of T cells, which T cells in turn can be used for passive transfer.

ABBREVIATIONS: CFA—complete Freund's adjuvant; CNS—central nervous system; EAE—experimental autoimmune encephalomyelitis; IFA—incomplete Freund's adjuvant; ISCI—incomplete spinal cord injury; MBP—myelin basic protein; MP—methylprednisolone; NS—nervous system; OVA—ovalbumin; PNS—peripheral nervous system.

### BACKGROUND OF THE INVENTION

The nervous system includes the CNS and the PNS. The CNS is composed of the brain and spinal cord; the PNS consists of all of the other neural elements, namely the nerves and ganglia outside of the brain and spinal cord.

Damage to the nervous system may result from a traumatic injury, such as penetrating trauma or blunt trauma, or a disease or disorder, including but not limited to Alzheimer's disease, Parkinson's disease, multiple sclerosis, Huntington's disease, amyotrophic lateral sclerosis (ALS), diabetic neuropathy, senile dementia, and ischemia.

Maintenance of CNS integrity is a complex “balancing act” in which compromises are struck with the immune system. In most tissues, the immune system plays an essential part in protection, repair, and healing. In the CNS, because of its unique immune privilege, immunological reactions are relatively limited. A growing body of evidence indicates that the failure of the mammalian CNS to achieve functional recovery after injury reflects an ineffective dialog between the damaged tissue and the immune system. For example, the restricted communication between the CNS and blood-borne macrophages affects the capacity of axotomized axons to regrow; transplants of activated macrophages can promote central nervous system regrowth (Rapalino et al., 1998).

Activated T cells have been shown to enter the CNS parenchyma, irrespective of their antigen specificity, but only T cells capable of reacting with a CNS antigen seem to persist there. T cells reactive to antigens of the CNS white matter, such as myelin basic protein (MBP), can induce the paralytic disease experimental autoimmune encephalomyelitis (EAE) within several days of their inoculation into naive recipient rats (Ben Nun and Cohen, 1982). Anti-MBP T cells may also be involved in the human disease multiple sclerosis (Ota et al., 1990). However, despite their pathogenic potential, anti-MBP T cell clones are present in the immune systems of healthy subjects (Burns et al., 1983). Activated T cells, which normally patrol the intact CNS, transiently accumulate at sites of CNS white matter lesions (Hirschberg et al., 1998).

A catastrophic consequence of CNS injury is that the primary damage is often compounded by the gradual secondary loss of adjacent neurons that apparently were undamaged, or only marginally damaged, by the initial injury. The primary lesion causes changes in extracellular ion concentrations, elevation of amounts of free radicals, release of neurotransmitters, depletion of growth factors, and local inflammation. These changes trigger a cascade of destructive events in the adjacent neurons that initially escaped the primary injury (Bazan et al., 1995). This secondary damage is mediated by activation of voltage-dependent or agonist-gated channels, ion leaks, activation of calcium-dependent enzymes such as proteases, lipases and nucleases, mitochondrial dysfunction and energy depletion, culminating in neuronal cell death. The widespread loss of neurons beyond the loss caused directly by the primary injury has been called “secondary degeneration.”

Another tragic consequence of CNS injury is that neurons in the mammalian CNS do not undergo spontaneous regeneration following an injury. Thus, a CNS injury causes permanent impairment of motor and sensory functions.

Spinal cord lesions, regardless of the severity of the injury, initially result in a complete functional paralysis known as spinal shock. Some spontaneous recovery from spinal shock may be observed, starting a few days after the injury and tapering off within three to four weeks. The less severe the insult, the better the functional outcome. The extent of recovery is a function of the amount of undamaged tissue minus the loss due to secondary degeneration. Recovery from injury would be improved by neuroprotective treatment that could reduce secondary degeneration.

Beneficial autoimmunity is a relatively new concept. It refers to a benign immune response that contributes to the maintenance and protection of injured neurons and the promotion of recovery after traumatic injury to the CNS (Moalem et al., 1999a; Schwartz and Cohen, 2000; and Schwartz et al., 1999). The pathological aspects of autoimmunity in the CNS, leading to various autoimmune syndromes, are well characterized. Recent findings suggest, however, that a benign immune response to injury-associated self-antigens may facilitate processes of tissue maintenance and wound healing, possibly by providing the damaged tissue with trophic factors (Moalem et al., 1999a; Hauben et al., 2000a; Hauben et al., 2000b; and Moalem et al., 2000). This response is reminiscent of the response evoked by pathogen attack, where recruitment of the immune system is considered essential. When the damage to the CNS is non-pathogenic, recruitment of the adaptive immune system has not been considered relevant, as there seems to be no obvious need to mount a defense. Surprisingly, however, it was found even with non-pathogenic damage such as that occurring in CNS trauma, an anti-self immune response is evoked, its purpose being to halt the progressive degeneration.

Progression of damage is a common occurrence after any CNS insult. Consequently, the outcome of spinal cord injury is far more severe than might be expected from the immediate effect of the insult. This is because the injury not only involves primary degeneration of the directly injured neurons, but also initiates a self-destructive process that leads to secondary degeneration of neighboring neurons that escaped the initial insult (Bazan et al., 1995). Much research has been devoted to limiting the extent of secondary degeneration and thereby improving functional recovery from partial CNS injury (Moalem et al., 1999a; Hauben et al., 2000b; Basso et al., 1996; Behrmann et al., 1994; Brewer et al., 1999).

The role of self-reactive lymphocytes known to be present in the blood of healthy individuals (Burns et al., 1983) is unclear. An increase in the numbers of myelin-reactive T cells following spinal cord contusion has been reported, but their function is controversial (Popovich et al., 1998). It was claimed that in Lewis rats such T cells might be destructive, as their transfer into naive animals led to symptoms of experimental autoimmune encephalomyelitis (EAE)(Popovich, 1996). Studies by our group, as well as by others, showed that after CNS injury endogenous myelin-associated T cells exert a physiological neuroprotective effect (Hammarberg et al., 2000). Passive transfer of MBP-stimulated splenocytes obtained from spinally contused Sprague-Dawley rats 7 or 14 days after the injury, or active immunization 7 days before the injury with myelin-associated antigens emulsified in incomplete Freund's adjuvant (IFA), was shown to boost this physiological beneficial autoimmune response and lead to improved functional recovery (Hauben et al., 2000a).

Boosting of autoimmunity, however, may be both a blessing and a curse. Thus, in selecting a protocol for immunization, the choice of a suitable self-antigen is complicated by the fact that the selected antigen may also have the potential for autoimmune destruction. The identity of the endogenous antigen which evokes the physiological immune neuroprotective response is not known. Moreover, in seeking an effective antigen to promote this response, a key question arises: how could such an antigen be used to boost an autoimmune response that will be neuroprotective but will not cause an autoimmune disease? It seems reasonable to suggest that non-encephalitogenic peptides derived from identified self-proteins will be promising candidates. However, because of the diversity of the HLA in humans, it is unlikely that a self-protein sequence can be found that will be universally non-encephalitogenic.

PCT International Publication No. WO 99/60021 of the present applicants describes compositions for preventing or inhibiting degeneration in the CNS or PNS for ameliorating the effects of injury or disease, comprising an NS-specific antigen such as MBP or NS-specific activated T cells. The application also mentions peptides derived from an NS-specific antigen which have a sequence comprised within the antigen sequence.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

It has now been found according to the present invention that encephalitogenic self-peptides derived from the sequences of CNS-specific antigens such as MBP become non-encephalitogenic by modification of their sequences and still recognize the T-cell receptor.

The present invention thus relates to a pharmaceutical composition for promoting nerve regeneration or reducing or inhibiting degeneration in the central nervous system or peripheral nervous system to ameliorate the effects of injury or disease, comprising a pharmaceutically acceptable carrier and an active ingredient selected from:

(a) a peptide obtained by modification of a self-peptide derived from a CNS-specific antigen, which modification consists in the replacement of one or more amino acid residues of the self-peptide by different amino acid residues, said modified CNS peptide still being capable of recognizing the T-cell receptor recognized by the self-peptide but with less affinity (hereinafter “modified CNS peptide”);

(b) a nucleotide sequence encoding a modified CNS peptide of (a);

(c) T cells activated by a modified CNS peptide of (a); and

(d) any combination of (a)-(c).

In a preferred embodiment, the CNS-specific antigen is MBP, and the modified CNS peptide (also called altered peptide) is derived from the residues 87-99 of the human MBP sequence, more preferably by replacement of the lysine residue 91 by glycine (G91) or by alanine (A91) or by replacement of the proline residue 96 by alanine (A96).

The present invention also provides a method for promoting nerve regeneration or for reducing or inhibiting neuronal degeneration in the CNS or PNS to ameliorate the deleterious effects of injury or disease by administering to a subject in need thereof an effective amount of the active ingredient in the composition according to the present invention.

It is shown herein in the application that immunization with myelin-associated antigens, even if performed after the injury, promotes functional recovery from spinal cord injury. Moreover, the choice of antigen and adjuvant determines the efficacy of the evoked neuroprotective response. In an attempt to reduce the risk of pathogenic autoimmunity while retaining the benefit of neuroprotection, we immunized rats, following spinal cord injury, with MBP-derived peptides whose pathogenic properties had been weakened by replacement of 1 amino acid in the T-cell receptor-binding site. Immunization with such altered peptide ligands immediately after spinal cord contusion led to a significant improvement in recovery, assessed by locomotor activity in an open field, retrograde labeling of the rubrospinal tracts, and diffusion-anisotropy MRI. Further optimization of non-pathogenic myelin-derived peptides can be expected to lead to the development of an effective immunization protocol as a therapeutic strategy to prevent complete paralysis following spinal cord injury.

### BRIEF DESCRIPTION OF THE DRAWINGS

FIG. 1. T-cell response to MBP. Seven days before spinal cord contusion, Lewis rats were immunized with MBP (100 μg/rat) emulsified in IFA or in an equal volume of CFA containing 5 mg/ml M. tuberculosis. Spleens were excised 3 or 14 days after the injury and splenocytes were cultured together with MBP, or with the non-self antigen ovalbumin (OVA), or without any antigen, or with concanavalin A. Three days after the injury (10 days after immunization), the proliferative response to MBP was significantly stronger in rats immunized with MBP in CFA than in rats immunized with MBP in IFA (p<0.05, 2-tailed Student's t-test). MBP-reactive T cells were detected in splenocytes excised and cultured 14 days after spinal contusion (21 days after immunization) in rats subjected to both MBP-immunization protocols, but the response was significantly stronger when CFA was used as adjuvant (p<0.05, 2-tailed Student's t-test).

FIGS. 2A-B. The type of adjuvant can affect the outcome of spinal cord injury. (A) Seven female Lewis rats, immunized (7 days before contusion) with spinal cord homogenate emulsified in CFA containing 0.5 mg/ml M. butyricum, recovered significantly better (p<0.05, 2-way ANOVA with replications;(*p<0.05; **p<0.01, 2-tailed Student's t-test) than 7 control littermates injected with PBS in the same adjuvant. (B) The same immunization protocol, when applied with a stronger adjuvant, has the opposite effect. Rats immunized with spinal cord homogenate in CFA containing 5 mg/ml M. tuberculosis developed severe EAE symptoms and showed significantly worse recovery than 6 control littermates injected with PBS in the same adjuvant (*p<0.05, 2-way ANOVA with replications).

FIG. 3. Retrograde labeling of cell bodies in the red nucleus. Three months after spinal contusion, preceded 7 days earlier by immunization with spinal cord homogenate emulsified in CFA (containing 0.5 mg/ml bacteria) or by injection with PBS in the same adjuvant (FIG. 2A), 2 rats from each group were re-anesthetized and the dye rhodamine dextran amine (Fluoro-ruby) was applied below the site of contusion. Five days later the rats were killed and their brains were excised, processed, and cryosectioned. Sections taken through the red nucleus were inspected and analyzed qualitatively and quantitatively by fluorescence and confocal microscopy. Significantly more labeled rubrospinal neurons were seen in slices from the immunized rats (right) (BBB score=8) than from the PBS-treated rats (left) (BBB score=5.5).

FIG. 4. Maps showing diffusion anisotropy of the contused spinal cords. Rats were deeply anesthetized and their excised spinal cords were immediately fixed and placed in 5-mm NMR tubes. The figure shows representative maps of spinal cords of rats immunized with spinal cord homogenate and control rats, after contusion at T8. Colors correspond to anisotropy ratios. The maps show the preservation of longitudinally ordered tissue at the lesion sites of the immunized rats. Note that the site of injury in the controls is much larger than in rats from the immunized group. The center of the injury site, determined by the slice with the lowest anisotropy value (290 arbitrary units for the immunized rats [BBB=8.5] and 167 units in the control rats [BBB=6]).

FIG. 5. Functional outcome of spinal cord contusion in an EAE-resistant strain can be improved by active immunization. Five male SPD rats were immunized with spinal cord homogenate (SCH) emulsified in CFA (containing 0.5 mg/ml bacteria) and 5 were injected with PBS in the same adjuvant. Twelve days later the rats were subjected to spinal cord contusion and their locomotor behavior in an open field was scored at the indicated times. Significantly better recovery was observed in the immunized rats than in the PBS-treated controls (p<0.05, 2-way ANOVA with replications; *p<0.05, 2-tailed Student's t-test).

FIG. 6. Immunization with myelin-associated proteins promotes neuroprotection of uninjured and partly injured fibers, but not axonal regeneration. Seven days before complete spinal cord transection, 5 female Lewis rats were immunized with spinal cord homogenate in IFA and 5 littermates were injected with PBS in IFA. Five female littermates were immunized directly after transection. None of these rats showed any significant locomotor function when tested up to 4 months after the injury, suggesting that although active immunization with spinal cord homogenate has a neuroprotective effect on axons that survived the direct injury, it does not lead to regeneration of completely transected axons.

FIG. 7. Immunization with a “safe”, non-encephalitogenic, modified MBP peptide can promote recovery from spinal cord contusion. Five female Lewis rats were immunized, immediately after spinal cord contusion, with G91 peptide (100 μg/rat) emulsified in CFA (containing 0.5 mg/ml bacteria). Five female littermates were subjected to spinal cord contusion and immediately injected with PBS in the same adjuvant. Significantly better functional recovery was observed in the immunized rats than in the PBS-treated controls (p<0.05, 2-way ANOVA with replications; *p<0.05, 2-tailed Student's t-test).

FIGS. 8A-B. Immunization following spinal cord injury with a modified MBP peptide (A96) promotes functional recovery in EAE-resistant SPD rats. (A) Six SPD male rats were immunized, immediately after spinal cord contusion, with A96 peptide (100 μg/rat) in CFA (containing 0.5 mg/ml bacteria), and 6 littermates were injected with PBS in the same adjuvant. Rats immunized with A96 recovered significantly better than PBS-injected controls (p<0.05, 2-way ANOVA with replications; *p<0., 2-tailed Student's t-test). (B) Immunization of SPD male rats (n=5), immediately after spinal cord contusion, with A96 peptide (500 μg/rat) in CFA (containing 0.5 mg/ml bacteria) had a significant negative effect on functional recovery (p<0.05, 2-way ANOVA with replications).

FIGS. 9a-d. Methylprednisolone (MP) obliterates the neuroprotective effect induced by A91 immunization. The figure shows open-field motor scores for Lewis rats (a) and Sprague-Dawley rats (b) treated with CFA+PBS (triangles), CFA+A91 (squares), MP+A91 (diamonds) or MP alone (circles). *, P=0.05, repeated ANOVA, mean ±s.e.m. Retrograde labeling of cell bodies in the red nuclei of Lewis rats (c) and SPD rats (d). *, P=0.05 for Lewis and 0.004 for SPD, Student t-test, A91 versus A91+MP, mean ±s.e.m. Significant reduction in motor recovery as well as in neuronal survival was observed in rats treated with a combination of A91 and MP.

FIGS. 10a-b. Recruitment of T cells and ED1-positive cells in the spinal cords of A91-immunized rats is reduced by MP. a, Accumulation of T cells at the site of injury in Lewis rats (black bars) and Sprague-Dawley rats (white bars). *, P=0.05, Student's t-test, mean ±s.e.m. b, Migration of ED-1 positive cells to the site of injury in Lewis rats (dark columns) and Sprague-Dawley rats (white columns). *, P<0.001, Student's t-test, mean ±s.e.m. MP significantly reduced the numbers of recruited cells at the site of injury in A91-immunized rats.

FIGS. 11a-c. Immediate or delayed vaccination with A91 promotes better motor recovery than that promoted by MP. a-c, Open-field motor score of Sprague-Dawley rats treated immediately (0) or 48 h postinjury with PBS (0)+CFA (48 h) (triangles); PBS (0)+A91 (48 h) (rectangles); MP alone (0)(circles); MP (0)+A91 (48 h) (diamonds); or A91 alone (0) (squares). For clarity, the five groups are presented more than once in the three panels. *, P<0.05, A91 versus CFA; **, P=0.05, PBS (0)+A91 (48 h) versus MP; ***, P=0.05, MP alone versus MP (0)+A91 (48 h); Student's t-test, mean ±s.e.m. The therapeutic window of the vaccine is apparently sufficient to allow promotion of motor recovery even if MP is administered immediately after the injury. The changes induced by MP appear to be partially reversible 48 h after injury.

FIGS. 12a-d. Cyclosporin-A (Cs-A), like MP, obliterates the neuroprotective effect evoked by immunization with A91. a-b, Open-field motor score of Lewis rats (a) and Sprague-Dawley rats (b) treated with CFA (triangles), A91 (squares), or A91+CsA (circles). *, P<0.05, repeated ANOVA, mean ±s.e.m. c-d, Retrograde labeling of cell bodies in the red nucleus of Lewis rats (c) and SPD rats (d). *, P=0.01, Student's t-test, A91 versus A91+CsA, mean ±s.e.m. Obliteration of the benefit of the therapeutic vaccination by anti-inflammatory agents argues in favor of modulation rather than suppression of the immune response after ISCI.

### DETAILED DESCRIPTION OF THE INVENTION

The outcome of spinal cord injury is far more severe than one might predict based on the immediate effect of the insult. This is so because the injury not only involves primary degeneration of the impacted neurons, but also spreads by a self-destructive process that leads to secondary degeneration of surrounding neurons that escaped the initial insult. Interestingly, however, concomitant with the onset of secondary degeneration, a spontaneous signal is transmitted systemically to the immune system where it evokes an adaptive immune response associated with nerve protection and maintenance.

This response is very similar to that evoked by pathogen attack, against which recruitment of the immune system is considered essential. In the context of non-pathogenic damage in the CNS, recruitment of the adaptive immune system has not been considered an issue, since there seems to be no obvious need to mount a defense. Surprisingly, however, the present inventors found that even with non-pathogenic damage, such as that occurring in CNS trauma, an anti-self immune response is evoked, with the purpose of halting the progression of damage. Passive and active immunization with self-antigens normally found in the body can have a therapeutic effect by boosting any endogenous immune response to damage.

The laboratory of the present inventors have recently discovered that passive or active immunization with T cells directed against CNS-specific myelin antigen or peptide derived from them reduces the post-traumatic spread of damage (Moalem et al., 1999a, 1999b; Hauben et al., 2000a, 2000b).

To derive the maximum to fully benefit from autoimmune neuroprotection, activated anti-self T cells used for immunization should be “safe”, i.e., they should be able to confer the benefit of protection without the accompanying risk of autoimmune disease. It is important to emphasize that unlike therapies for autoimmune disease, which are based on immune deviation, or tolerance, or response even from general immunosuppression, immune neuroprotective therapy is based on active T cell anti-self response which is insufficiently effective in its spontaneous form and is therefore in need of boosting.

The concept of the present invention lies on the finding that the ideal approach seems to be the use of a modified peptide that is immunogenic but not encephalitogenic. The most suitable peptides for this purpose are those in which an encephalitogenic self-peptide is modified at the T cell receptor (TCR) binding site and not at the MHC binding site(s), so that the immune response is activated but not anergized (Karin et al., 1998; Vergelli et al., 1996).

Thus, the present invention provides pharmaceutical compositions comprising an antigen being a synthetic modified CNS peptide for reducing or inhibiting the effects of injury or disease that result in nervous system degeneration or for promoting nerve regeneration in the nervous system, particularly in the CNS. Additionally, these modified CNS peptides may be used for in vivo or in vitro activation of T cells.

In another embodiment, methods for promoting nerve regeneration or for reducing or inhibiting the effects of CNS or PNS injury or disease involve administering a synthetic modified CNS peptide antigen to activate T cells in vivo and thereby produce a population of T cells that accumulate at a site of injury or disease of the CNS or PNS.

The modified CNS peptide may be produced by modification of a self-peptide derived from a CNS-specific antigen such as, but not being limited to, myelin basic protein (MBP), myelin oligodendrocyte glycoprotein (MOG), proteolipid protein (PLP), myelin-associated protein (MAG), S-100, β-amyloid, Thy-1, P0, P2 and any other nervous system-specific protein in which one or more amino acids in their TCR binding site was altered. The modified peptide has from 9 to 20, preferably 9-13, amino acid residues.

In a preferred embodiment, the CNS-specific antigen is MBP and the self-peptide is selected from the peptides p11, p51-70, p87-99, p91-110, p131-150, or p151-170 of MBP.

In a more preferred embodiment, the modified CNS peptide according to the invention includes, but is not limited to a peptide derived from the residues 87-99 of human MBP (SEQ ID NO:1; Genbank accession number 307160; Kamholz et al., 1986), in which the lysine residue 91 is replaced by glycine (G91) (SEQ ID NO:2) or by alanine (A91) (SEQ ID NO:3) or the proline residue 96 is replaced by alanine (A96)) (SEQ ID NO:4) (Karin et al., 1998). Peptide analogues derived from the residues 86 to 99 of human MBP by alteration of positions 91, 95 or 97 have been disclosed in U.S. Pat. No. 5,948,764 for treatment of multiple sclerosis.

In addition, any encephalitogenic epitopes in which critical amino acids in their TCR binding site but not MHC binding site are altered are encompassed by the present invention as long as they are non-encephalitogenic and still recognize the T-cell receptor.

Activated T cells with the modified CNS peptides of the invention can also be used for ameliorating or inhibiting the effects of injury or disease of the CNS or PNS that result in nerve degeneration or for promoting regeneration in the nervous system, in particular the CNS.

To minimize secondary damage after nerve injury, patients can be treated by administering autologous or semi-allogeneic T lymphocytes sensitized to at least one modified CNS peptide of the invention. As the window of opportunity has not yet been precisely defined, therapy should be administered as soon as possible after the primary injury to maximize the chances of success, preferably within about one week.

To bridge the gap between the time required for activation and the time needed for treatment, a bank can be established with personal vaults of autologous T lymphocytes prepared for future use for neuroprotective therapy against secondary degeneration in case of CNS injury. T lymphocytes are isolated from the blood and then sensitized to a modified CNS peptide antigen. The cells are then frozen and suitably stored under the person's name, identity number, and blood group, in a cell bank until needed.

Additionally, autologous stem cells of the CNS can be processed and stored for potential use by an individual patient in the event of traumatic disorders of the CNS such as ischemia or mechanical injury, as well as for treating neurodegenerative conditions such as Alzheimer's disease or Parkinson's disease. Alternatively, semi-allogeneic or allogeneic T cells can be stored frozen in banks for use by any individual who shares one MHC type II molecule with the source of the T cells.

The T cells activated by the modified CNS peptide are preferably autologous, most preferably of the CD4 and/or CD8 phenotypes, but they may also be allogeneic T cells from related donors, e.g., siblings, parents, children, or HLA-matched or partially matched, semi-allogeneic or fully allogeneic donors.

In addition to the use of autologous T cells isolated from the subject, the present invention also comprehends the use of semi-allogeneic T cells for neuroprotection. These T cells may be prepared as short- or long-term lines and stored by conventional cryopreservation methods for thawing and administration, either immediately or after culturing for 1-3 days, to a subject suffering from injury to the central nervous system and in need of T cell neuroprotection.

The use of semi-allogeneic T cells is based on the fact that T cells can recognize a specific antigen epitope presented by foreign antigen-presenting cells (APC), provided that the APC express the MHC molecule, class I or class II, to which the specific responding T cell population is restricted, along with the antigen epitope recognized by the T cells. Thus, a semi-allogeneic population of T cells that can recognize at least one allelic product of the subject's MHC molecules, preferably an HLA-DR or an HLA-DQ or other HLA molecule, and that is specific for a modified CNS peptide-associated antigen epitope, will be able to recognize such antigen in the subject's area of CNS damage and produce the needed neuroprotective effect. There is little or no polymorphism in the adhesion molecules, leukocyte migration molecules, and accessory molecules needed for the T cells to migrate to the area of damage, accumulate there, and undergo activation. Thus, the semi-allogeneic T cells will be able to migrate and accumulate at the CNS site in need of neuroprotection and will be activated to produce the desired effect.

It is known that semi-allogeneic T cells will be rejected by the subject's immune system, but that rejection requires about two weeks to develop. Hence, the semi-allogeneic T cells will have a two week window of opportunity needed to exert neuroprotection. After two weeks, the semi-allogeneic T cells will be rejected from the body of the subject, but that rejection is advantageous to the subject because it will rid the subject of the foreign T cells and prevent any untoward consequences of the activated T cells. The semi-allogeneic T cells thus provide an important safety factor and are a preferred embodiment.

It is known that a relatively small number of HLA class II molecules are shared by most individuals in a population. For example, about 50% of the Jewish population express the HLA-DR5 gene. Thus, a bank of specific T cells reactive to APL antigen epitopes that are restricted to HLA-DR5 would be useful in 50% of that population. The entire population can be covered essentially by a small number of additional T cell lines restricted to a few other prevalent HLA molecules, such as DR1, DR4, DR2, etc. Thus, a functional bank of uniform T cell lines can be prepared and stored for immediate use for almost any individual in a given population. Such a bank of T cells would overcome any technical problems in obtaining a sufficient number of specific T cells from the subject in need of neuroprotection during the open window of treatment opportunity. The semi-allogeneic T cells will be safely rejected after accomplishing their role of neuroprotection. This aspect of the invention does not contradict, and is in addition to, the use of autologous T cells as described herein.

The activated T cells of the invention are preferably non-attenuated, although attenuated APL-specific activated T cells may be used. T cells may be attenuated using methods well known in the art, including but not limited to, gamma-irradiation, e.g., 1.5-10.0 Rads (Ben-Nun and Cohen, 1982); and/or by pressure treatment, for example as described in U.S. Pat. No. 4,996,194; and/or chemical cross-linking with an agent such as formaldehyde, glutaraldehyde and the like, for example as described in U.S. Pat. No. 4,996,194; and/or cross-linking and photoactivation with light with a photoactivatable psoralen compound, for example as described in U.S. Pat. No. 5,114,721; and/or a cytoskeletal disrupting agent such as cytochalsin and colchicine, for example as described in U.S. Pat. No. 4,996,194. In a preferred embodiment, the APL-specific activated T cells are isolated as described below. T cells can be isolated and purified according to methods known in the art (Mor and Cohen, 1995).

Circulating T cells of a subject which recognize MBP or another CNS antigen such as the amyloid precursor protein, are isolated and expanded using known procedures. In order to obtain the activated T cells, T cells are isolated and the T cells activated by the modified CNS peptide are then expanded by a known procedure (Burns et al., 1983).

The isolated T cells may be activated by exposure of the cells to one or more of a variety of synthetic CNS-specific antigens or epitopes. During ex vivo activation of the T cells, the T cells may be activated by culturing them in medium to which at least one suitable growth promoting factor has been added. Growth promoting factors suitable for this purpose include, without limitation, cytokines, for instance TNF-α, IL-2 or IL-4.

In one embodiment, the activated T cells endogenously produce a substance that ameliorates the effects of injury or disease in the CNS.

In another embodiment, the activated T cells endogenously produce a substance that stimulates other cells, including, but not limited to, transforming growth factor-β (TGF-β), nerve growth factor (NGF), neurotrophic factor 3(NT-3), neurotrophic factor ⅘ (NT-⅘), brain derived neurotrophic factor (BDNF); interferon-γ (IFN-γ), and interleukin-6 (IL-6), wherein the other cells, directly or indirectly, ameliorate the effects of injury or disease.

Following their proliferation in vitro, the T cells are administered to a mammalian subject. In a preferred embodiment, the T cells are administered to a human subject. T cell expansion is preferably performed using peptides corresponding to sequences in a non-pathogenic, modified CNS peptide-specific, self protein.

A subject can initially be immunized with an CNS-specific antigen using a non-pathogenic peptide of the self protein. A T cell preparation can be prepared from the blood of such immunized subjects, preferably from T cells selected for their specificity towards the modified CNS peptide-specific antigen. The selected T cells can then be stimulated to and Cohen, 1982).

The activated T cells of the invention can be used immediately or may be preserved for later use, e.g., by cryopreservation as described below. Said activated T cells may also be obtained using previously cryopreserved T cells, i.e., after thawing the cells, the T cells may be incubated with the modified CNS peptide, optimally together with thymocytes.

As will be evident to those skilled in the art, the T cells can be preserved, e.g., by cryopreservation, either before or after culture.

Cryopreservation agents which can be used include, but are not limited to, dimethyl sulfoxide (DMSO), glycerol, polyvinylpyrrolidone, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, inorganic salts, and DMSO combined with hydroxyethyl starch and human serum albumin.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve.

After thorough freezing, cells can be rapidly transferred to a long-term cryogenic storage vessel. In one embodiment, samples can be cryogenically stored in mechanical freezers, such as freezers that maintain a temperature of about −80° C. or about −20° C. In a preferred embodiment, samples can be cryogenically stored in liquid nitrogen (−196° C.) or its vapor. Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum. Other methods of cryopreservation of viable cells, or modifications thereof, are available and envisioned for use, e.g., cold metal-mirror techniques.

Frozen cells are preferably thawed quickly (e.g., in a water bath maintained at 37-47° C.) and chilled immediately upon thawing. It may be desirable to treat the cells in order to prevent cellular clumping upon thawing. To prevent clumping, various procedures can be used, including but not limited to the addition before or after freezing of DNAse, low molecular weight dextran and citrate, citrate, hydroxyethyl starch, or acid citrate dextrose.

The cryoprotective agent, if toxic in humans, should be removed prior to therapeutic use of the thawed T cells. One way in which to remove the cryoprotective agent is by dilution to an insignificant concentration.

Once frozen T cells have been thawed and recovered, they are used to promote neuronal regeneration as described herein with respect to non-frozen T cells. Once thawed, the T cells may be used immediately, assuming that they were activated prior to freezing. Preferably, however, the thawed cells are cultured before injection to the patient in order to eliminate non-viable cells. Furthermore, in the course of this culturing over a period of about one to three days, an appropriate activating agent can be added so as to activate the cells, if the frozen cells were resting T cells, or to help the cells achieve a higher rate of activation if they were activated prior to freezing. Usually, time is available to allow such a culturing step prior to administration as the T cells may be administered as long as a week after injury, and possibly longer, and still maintain their neuroregenerative and neuroprotective effect.

The pharmaceutical compositions according to the present invention may be used to promote nerve regeneration or to reduce or inhibit secondary degeneration which may otherwise follow primary CNS injury, e.g., blunt trauma, penetrating trauma, hemorrhagic stroke, ischemic stroke or damages caused by surgery such as tumor excision. In addition, such compositions may be used to ameliorate the effects of disease that result in a degenerative process, e.g., degeneration occurring in either gray or white matter (or both) as a result of various diseases or disorders, including, without limitation: diabetic neuropathy, senile dementias, Alzheimer's disease, Parkinson's Disease, facial nerve (Bell's) palsy, glaucoma, Huntington's chorea, amyotrophic lateral sclerosis (ALS), non-arteritic optic neuropathy, intervertebral disc herniation, vitamin deficiency, prion diseases such as Creutzfeldt-Jakob disease, carpal tunnel syndrome, peripheral neuropathies associated with various diseases, including but not limited to, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, complications of various drugs (e.g., metronidazole) and toxins (e.g., alcohol or organophosphates), Charcot-Marie-Tooth disease, ataxia telangectasia, Friedreich's ataxia, amyloid polyneuropathies, adrenomyelo-neuropathy, Giant axonal neuropathy, Refsum's disease, Fabry's disease, lipoproteinemia, etc.

In a preferred embodiment, the modified CNS peptides, the nucleotide sequences encoding them or the T cells activated therewith, or any combination thereof of the present invention are used to treat diseases or disorders where promotion of nerve regeneration, or reduction or inhibition of secondary neural degeneration, is indicated, which are not autoimmune diseases or neoplasias. In a preferred embodiment, the compositions of the present invention are administered to a human subject.

While activated T cells may have been used in the prior art in the course of treatment to develop tolerance to autoimmune antigens in the treatment of autoimmune diseases, or in the course of immunotherapy in the treatment of CNS neoplasms, the present invention can also be used to ameliorate the degenerative process caused by autoimmune diseases or neoplasms as long as it is used in a manner not suggested by such prior art methods. Thus, for example, T cells activated by an autoimmune antigen have been suggested for use to create tolerance to the autoimmune antigen and, thus, ameliorate the autoimmune disease. Such treatment, however, would not have suggested the use of T cells activated by modified CNS peptides which will not induce tolerance to the autoimmune antigen, or the use of T cells which are administered in such a way as to avoid creation of tolerance. Similarly, for neoplasms, the effects of the present invention can be obtained without using immunotherapy processes suggested in the prior art by, for example, using an APL antigen which does not appear in the neoplasm. T cells activated with such an antigen will still accumulate at the site of neural degeneration and facilitate inhibition of this degeneration, even though it will not serve as immunotherapy for the tumor per se.

Pharmaceutical compositions for use in accordance with the present invention may be formulated in a conventional manner using one or more physiologically acceptable carriers or excipients. The carrier(s) must be “acceptable” in the sense of being compatible with the other ingredients of the composition and not deleterious to the recipient thereof.

The term “carrier” refers to a diluent, adjuvant, excipient, or vehicle with which the therapeutic is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulfate; a glidant, such as colloidal silicon dioxide; a sweetening agent, such as sucrose or saccharin; and/or a flavoring agent, such as peppermint, methyl salicylate, or orange flavoring.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well-known in the art.

Preparations for oral administration may be suitably formulated to give controlled release of the active compound.

For buccal administration, the compositions may take the form of tablets or lozenges formulated in conventional manner.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

The compositions may also be formulated in rectal compositions such as suppositories or retention enemas, e.g., containing conventional suppository bases such as cocoa butter or other glycerides.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoro-methane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

In a preferred embodiment, compositions comprising a modified CNS peptide according to the invention, T cells activated thereby or a nucleotide sequence encoding such peptide, are formulated in accordance with routine procedures as pharmaceutical compositions adapted for intravenous or intraperitoneal administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

Pharmaceutical compositions comprising a modified CNS peptide may optionally be administered with an adjuvant.

The invention also provides a pharmaceutical pack or kit comprising one or more containers filled with one or more of the ingredients of the pharmaceutical compositions of the invention.

In a preferred embodiment, the pharmaceutical compositions of the present invention are administered to a mammal, preferably a human, shortly after injury or detection of a degenerative lesion in the CNS. The therapeutic methods of the invention may comprise administration of a modified CNS peptide, a nucleotide sequence encoding such peptide, or T-cells activated therewith, or any combination thereof. When using combination therapy, the peptide may be administered before, concurrently or after administration of the activated T cells or of the nucleotide encoding such peptide.

In one embodiment, the compositions of the invention are administered in combination with one or more of the following (a) mononuclear phagocytes, preferably cultured monocytes (as described in PCT publication No. WO 97/09985, which is incorporated herein by reference in its entirety), that have been stimulated to enhance their capacity to promote neuronal regeneration; (b) a neurotrophic factor such as acidic fibroblast growth factor.

In another embodiment, mononuclear phagocyte cells according to PCT Publication No. WO 97/09985 and U.S. Pat. No. 6,267,955, are injected into the site of injury or lesion within the CNS, either concurrently, prior to, or following parenteral administration of a modified CNS peptide, nucleotide sequence or activated T cells according to the invention.

In a further embodiment, a modified CNS peptide, nucleotide sequence or activated T cells according to the invention may be administered as a single dose or may be repeated, preferably at 2 week intervals and then at successively longer intervals such as once a month, once a quarter, once every six months, etc. The course of treatment may last several months, several years or occasionally also through the lifetime of the individual, depending on the condition or disease which is being treated. In the case of a CNS injury, the treatment may range between several days to months or even years, until the condition has stabilized and there is no risk or only a limited risk of developing secondary degeneration. In chronic human disease or Parkinson's disease, the therapeutic treatment in accordance with the invention may be for life.

As will be evident to those skilled in the art, the therapeutic effect depends at times on the condition or disease to be treated, on the individual's age and health condition, on other physical parameters (e.g., gender, weight, etc.) of the individual, as well as on various other factors, e.g., whether the individual is taking other drugs, etc.

The optimal dose of the therapeutic compositions comprising the activated T cells of the invention is proportional to the number of nerve fibers affected by CNS injury or disease at the site being treated. In a preferred embodiment, the dose ranges from about 5×106 to about 107 for treating a lesion affecting about 105 nerve fibers, such as a complete transection of a rat optic nerve, and ranges from about 107 to about 108 for treating a lesion affecting about 106-107 nerve fibers, such as a complete transection of a human optic nerve. As will be evident to those skilled in the art, the dose of T cells can be scaled up or down in proportion to the number of nerve fibers thought to be affected at the lesion or site of injury being treated.

Having now generally described the invention, the same will be more readily understood through reference to the following example which is provided by way of illustration and is not intended to be limiting of the present invention.

### EXAMPLES

### Materials and Methods

Animals. Inbred adult Lewis or Sprague-Dawley (SPD) rats (10-12 weeks old, 200-250 g, or 13-14 weeks old, 180-220 g) were supplied by the Animal Breeding Center of The Weizmann Institute of Science, Rehovot, Israel. The rats were matched for age and weight in each experiment and housed in a light- and temperature-controlled room.

Antigens. MBP was prepared from the spinal cords of guinea pigs as previously described (Moalem et al., 1999a), or purchased from Sigma (St. Louis, Mo.). Spinal cord homogenate was prepared from autologous rat spinal cords homogenized in phosphate-buffered saline (PBS) (vol/vol). Modified (non-encephalitogenic) MBP peptides were derived from an encephalitogenic peptide, amino acids 87-99 of MBP, by replacing the lysine residue 91 with glycine (G91, kindly donated by Prof. L. Steinman) or with alanine (A91) or the proline residue 96 with alanine (A96) (A91 and A96 were synthesized at the Weizmann Institute of Science, Rehovot, Israel). All peptides used in the study had a purity of >95% as confirmed by reverse-phase HPLC. Antigens were emulsified in equal volumes of IFA (Difco, Detroit, Mich.), CFA supplemented with 5 mg/ml Mycobacterium tuberculosis (Difco, Detroit, Mich.) (an amount which in uninjured rats leads to severe EAE symptoms), or CFA with a low bacterial supplement (0.5 mg/ml M. butyricum, Difco).

Spinal cord contusion or transection. One group of rats were anesthetized by intraperitoneal injection of Rompun (xylazine, 10 mg/kg; Vitamed, Israel) and Vetalar (ketamine, 50 mg/kg; Fort Dodge Laboratories, Fort Dodge, Iowa) and their spinal cords were exposed by laminectomy at the level of T8. Another group of rats were anesthetized by intramuscular injection of Chanazine (xylazine, 10 mg/kg; Chanelle Phamaceuticals, Longhrea, Ireland) and Vetalar as above and their spinal cords were exposed by laminectomy at the level of T9. One hour after induction of anesthesia, a 10-g rod was dropped onto the laminectomized cord from a height of 50 mm, using the NYU impactor, a device shown to inflict a well-calibrated contusive injury of the spinal cord (Basso et al., 1996). The spinal cords of another group of rats were completely transected, as previously described (Rapalino et al., 1998).

Drugs. Sodium succinate MP (30 mg/kg, Solu-Medrol, Pharmacia & Upjohn, Puurs, Belgium) was injected into the tail vein in one or several doses after ISCI. Cyclosporin-A (3 mg/kg), Novartis, Basel, Switzerland) was injected intraperitoneally immediately after and twice more at 12-hour intervals after injury. Control rats received injections of saline only.

Active immunization. Rats were immunized subcutaneously, on a random basis, with MBP, spinal cord homogenate, or modified MBP peptide, or injected with PBS, each emulsified in an equal volume of CFA containing 5 mg/ml M. tuberculosis, CFA containing 0.5 mg/ml M. butyricum, or IFA. The immunization was performed within 1 hour after contusion or 7 days earlier. Control rats were sham-operated (laminectomized but not contused), immunized, and examined daily for symptoms of EAE, which were scored on a scale of 1 to 5 (Basso et al., 1995).

Animal care. In contused rats, bladder expression was assisted by massage at least twice a day (particularly during the first 48 h after injury, when it was done 3 times a day), until the end of the second week, by which time automatic voidance had been recovered in SPD rats. Lewis rats required this treatment throughout the experiment. All rats were carefully monitored for evidence of urinary tract infection or any other sign of systemic disease. During the first week after contusion and in any case of hematuria after this period, they received a course of sulfamethoxazole (400 mg/ml) and trimethoprim (8 mg/ml) (Resprim, Teva Laboratories, Israel), administered orally with a tuberculin syringe (0.3 ml of solution per day). Daily inspections included examination of the laminectomy site for evidence of infection and assessment of the hind limbs for signs of autophagia or pressure.

Proliferation assay. Seven days before spinal cord contusion, Lewis rats were immunized with MBP (100 μg/rat) emulsified in an equal volume of IFA or of CFA containing 5 mg/ml M. tuberculosis. Spleens were excised 3 or 14 days after the injury and pressed through a fine wire mesh. The washed splenocytes (2×106 cells/ml) were cultured in triplicate in flat-bottomed microtiter wells in 0.2 ml of proliferation medium containing Dulbecco's modified Eagle's medium (DMEM) supplemented with L-glutamine (2 mM), 2-mercaptoethanol (5×10-5 M), sodium pyruvate (1 mM), penicillin (100 IU/ml), streptomycin (100 μg/ml), non-essential amino acids, and autologous rat serum 1% (vol/vol). The cells were activated for 72 h at 37° C., 90% relative humidity and 7% CO2 in the presence of irradiated thymocytes (2000 rad, 2×106 cells/ml), together with MBP (15 μg/ml) or the non-self antigen OVA (15 μg/ml) or concanavalin A (1.25 μg/ml) or without any antigen. In another experiment, lymph node cells, excised and pooled 10 days after ISCI (n=3), were cultured in quadruplicate in 0.2 ml DMEM as above. The cells (2×105 cells per well) were cultured alone (no antigen) or together with MBP (15 μg/ml), OVA (15 μg/ml), A91 (15 μg/ml) or Con A (1.25 μg/ml). The proliferative response was determined by measuring the incorporation of 3-[H]thymidine (1 μCi/well), which was added for the last 16 h of a 72-h culture. The stimulation index was calculated by dividing the mean value (in cpm) of experimental wells by the mean value (in cpm) of the cells cultured in medium alone.

Assessment of recovery from spinal cord contusion. Behavioral recovery was scored in an open field using the Basso, Beattie, and Bresnahan locomotor rating scale (BBB)(Basso et al., 1995), where a score of 0 registers complete paralysis and a score of 21 complete mobility (Hauben et al., 2000a; Hauben et al., 2000b; and Basso et al., 1996). Blind scoring ensured that observers were not aware of the treatment received by individual rats. Approximately once a week, the locomotor activities of the trunk, tail, and hind limbs were evaluated in an open field by placing each rat for 4 min in the center of a circular enclosure (90 cm diameter, 7 cm wall height) made of molded plastic with a smooth, non-slip floor. Prior to each evaluation the rats were examined carefully for perineal infection, wounds in the hind limbs, and tail and foot autophagia.

Retrograde labeling of rubrospinal neurons. Three months after spinal contusion preceded by immunization with spinal cord homogenate emulsified in CFA (0.5 mg/ml bacteria) or by injection with PBS in the same adjuvant, 2 rats from each group were re-anesthetized and the dye rhodamine dextran amine (Fluoro-ruby, Molecular Probes, Eugene, Oreg.) was applied below the site of contusion at T12. After 5 days, the rats were again deeply anesthetized and their brains were excised, processed, and cryosectioned. Sections taken through the red nucleus were inspected and analyzed qualitatively and quantitatively by fluorescence and confocal microscopy. The total numbers of labeled cells were counted in every section from each brain. Thus, the number of labeled cells recorded for each brain is the sum of all the cells counted in each section. The number of labeled neurons in each rat is given by the average number of cells counted in its two red nuclei. In the statistical analysis, we used a corrective factor to allow for the thickness of the sections, and the size of a single nucleus to correct for possible recounting of the same cell (Smolen et al., 1983).

Diffusion-anisotropy magnetic resonance imaging (MRI). Diffusion anisotropy was measured in a Bruker DMX 400 widebore spectrometer, using a microscopy probe with a 5-mm Helmholz coil and actively shielded magnetic field gradients. The observer was blinded to each rat's identity. Multislice echo imaging was performed with 9e axial slices, with the central slice positioned at the center of the spinal injury. Images were obtained with a TE of 31 ms, TR 2000 ms, diffusion time 15 ms, diffusion gradient duration 3 ms, field of view 0.6 mm, matrix size 128×128 pixels, slice thickness 0.5 mm, and slice separation 1.18 mm. Left to right images represent axial sections from head to foot. Four diffusion gradient values (0, 28, 49, and 71 g/cm) were applied along the read direction (transverse diffusion) or along the slice direction (longitudinal diffusion). Using an exponential fit for each pixel, we obtained a transverse (T) and a longitudinal (L) apparent diffusion coefficient (ADC) map, from which an anisotropy ratio matrix was derived. The accumulated anisotropy in each slice was integrated. For each rat, the lowest value of the slice anisotropy integral was defined as the lesion site.

Immunohistochemistry. Seven days after ISCI, each rat was perfused intracardially and prepared for immunostaining studies as described previously (Butovsky et al., 2001). Briefly, after perfusion the spinal cords were removed, postfixed overnight and transferred to sucrose 30% for cryoprotection for at least 3 days. A 20-mm block of the spinal cord, with the injured site at the center, was excised and embedded in Tissue-Tek (Miles, Elkhart, Ind.). Frozen longitudinal 20-mm blocks were sectioned (20 μm). From each rat and for each staining, two tissue sections from the periphery (one from each side) and one from the epicenter were incubated for 1 h with the monoclonal antibody ED-1 (1:200; Serotec, Oxford, UK) for labeling of activated macrophages and microglia or anti-rat T cell receptor (TCR-α/β, 1:50, Serotec, UK) for detection of T cells. After rinsing, sections were incubated with the secondary antibody, FITC-conjugated goat anti-mouse (1:200; Jackson ImmunoResearch, West Grove, Pa.), for 1 h at room temperature. They were then prepared to be examined under a Zeiss laser-scanning confocal microscope (LSM510) and/or a Zeiss Axioplane 100 fluorescence light microscope. The results were analyzed by an observer who was blinded to the treatment received by the rats.

### Example 1

### T-cell Response to MBP after Immunization with Potent CFA

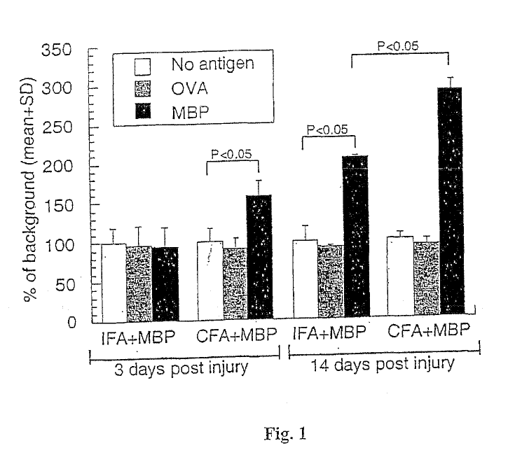

We have previously demonstrated that active immunization, 7 days before spinal cord contusion, with MBP emulsified in IFA leads to a reduction in the post-traumatic loss of neural tissue in Lewis rats, thereby improving functional recovery (Hauben et al., 2000a). This adjuvant was chosen on the assumption that it would promote a cell-mediated immune response but would not cause disease (Killen et al., 1982; Namikawa et al., 1982). In the present experiment, we first examined whether active immunization with MBP, immediately after contusion rather than before it, can effectively replace active immunization with MBP applied 7 days prior to contusion. Immunization with MBP emulsified in IFA, performed directly after severe spinal cord contusion, led to better recovery than that seen in control rats similarly injected with PBS in IFA. However, this post-injury immunization was not as effective as immunization with the same emulsion 7 days prior to the insult (Table 1). We reasoned that the difference was due to the delayed onset of the response to MBP relative to the therapeutic window for neuroprotection following spinal cord contusion (Hauben et al., 2000a). We therefore performed a set of experiments to compare the effects of IFA and the potent CFA, used as adjuvants for immunization, on the specific T-cell response to MBP. Seven days before spinal cord contusion, Lewis rats were immunized with MBP emulsified in IFA or CFA (5 mg/ml M. tuberculosis). Spleens were excised 3 or 14 days after the injury, and splenocyte proliferation was assayed (see Methods). Three days after the injury (10 days after immunization), a response to MBP could be detected in CFA-immunized rats, but not in IFA-immunized rats (FIG. 1). Fourteen days after the injury (21 days after immunization) a response to MBP was detectable in both CFA- and IFA-immunized rats, but the response in the CFA-immunized rats was significantly greater. In all examined rats, the response to the non-self antigen ovalbumin was similar to the background (no antigen) response (FIG. 1). These findings suggest that potent CFA induces an earlier and stronger T-cell response than that induced by IFA.

### Example 2

### Active Immunization with Spinal Cord Homogenate Emulsified in CFA

To determine whether the effectiveness of active immunization could be increased by a change in the protocol, we examined the effects of immunization with spinal cord homogenate, which contains a spectrum of myelin proteins, rather than with MBP only. In view of the results presented in FIG. 1, the adjuvant chosen for the following experiments was CFA with 2 different concentrations of bacterial component. Seven days before spinal cord contusion, female Lewis rats (n=7) were immunized with spinal cord homogenate emulsified in CFA (containing 0.5 mg/ml bacteria). A control group of female Lewis rats (n=7) was injected with PBS emulsified in the same adjuvant. Three non-injured female rats that were immunized according to the same protocol showed no detectable symptoms of EAE. In a separate set of experiments, female rats (n=6 for each group) were immunized, 7 days before spinal cord contusion, with spinal cord homogenate emulsified in CFA containing 5 mg/ml bacteria.

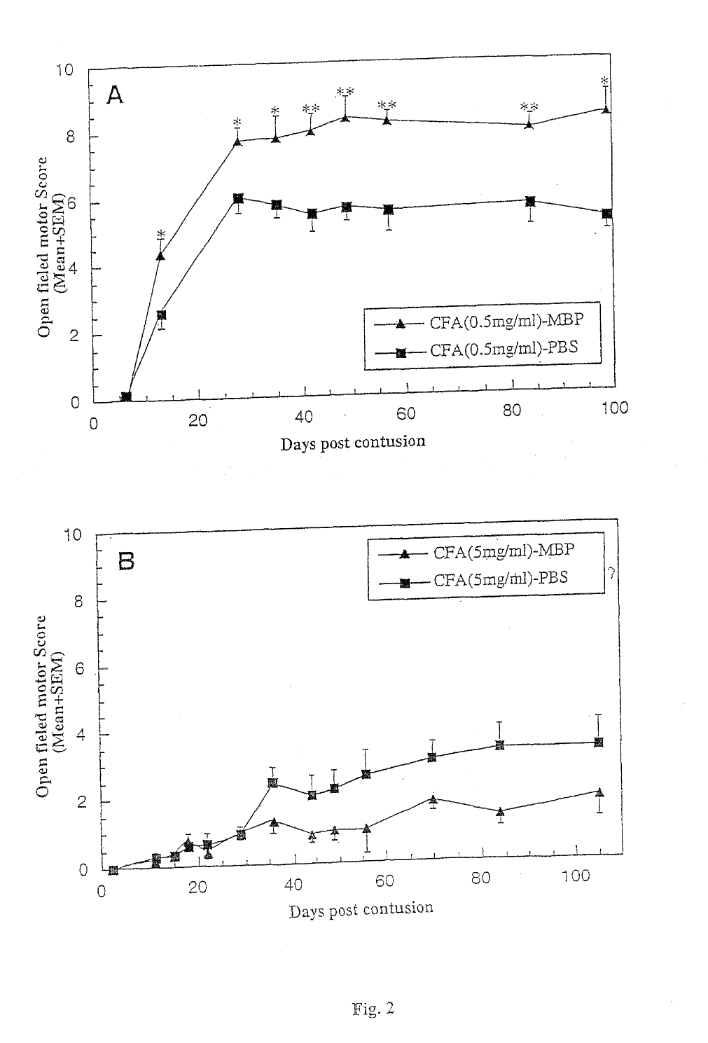

Immunization of female Lewis rats with spinal cord homogenate emulsified in the adjuvant with the lower bacterial content (0.5 mg/ml) resulted in significantly better recovery than that obtained in PBS-treated controls (FIG. 2A; maximal score of 8.2±0.2 [mean ±SE] on the BBB scale compared with 5.5±0.2 (2-way ANOVA with replications, p<0.05). A BBB score of 8.2 indicates extensive movement of all 3 hindlimb joints and plantar placement of the paw (3 animals showed occasional weight support and plantar steps), whereas a score of 5.5 indicates slight movement of 2 hindlimb joints and extensive movement of the third, without plantar placement of the paw or swiping and without weight support. After immunization with spinal cord homogenate emulsified in the more potent adjuvant (containing 5 mg/ml bacteria, FIG. 2B), spinally contused rats (n=6) showed no recovery relative to controls, and 3 non-injured littermates showed extremely severe symptoms of EAE. It should be noted that the control groups immunized with PBS in the 2 different adjuvants showed differences that might reflect a general effect of the immune response to trauma, possibly with some boosting by the bacteria. The observed loss of the beneficial effect, together with signs of severe encephalitogenicity, when the bacterial dosage was high raises questions about the connection between beneficial autoimmunity and development of autoimmune disease. It is possible that the mechanisms leading to protective and pathogenic autoimmunity are similar, and that the 2 types of autoimmune responses are mediated by the same T-cell type, which acts protectively or destructively depending on its dosage. Alternatively, it is possible that the 2 types of autoimmune responses are mediated by different populations of regulatory and pathogenic T cells, whose proportions determine the outcome of the injury. In either case, the present results suggest that the autoimmune response must be rigorously controlled in order to avoid pathogenicity and promote neuroprotection.

### Example 3

### Spinal Cord Preservation by Active Immunization Confirmed by Retrograde Labeling of Rubrospinal Neurons and by Diffusion-Anisotropy MRI

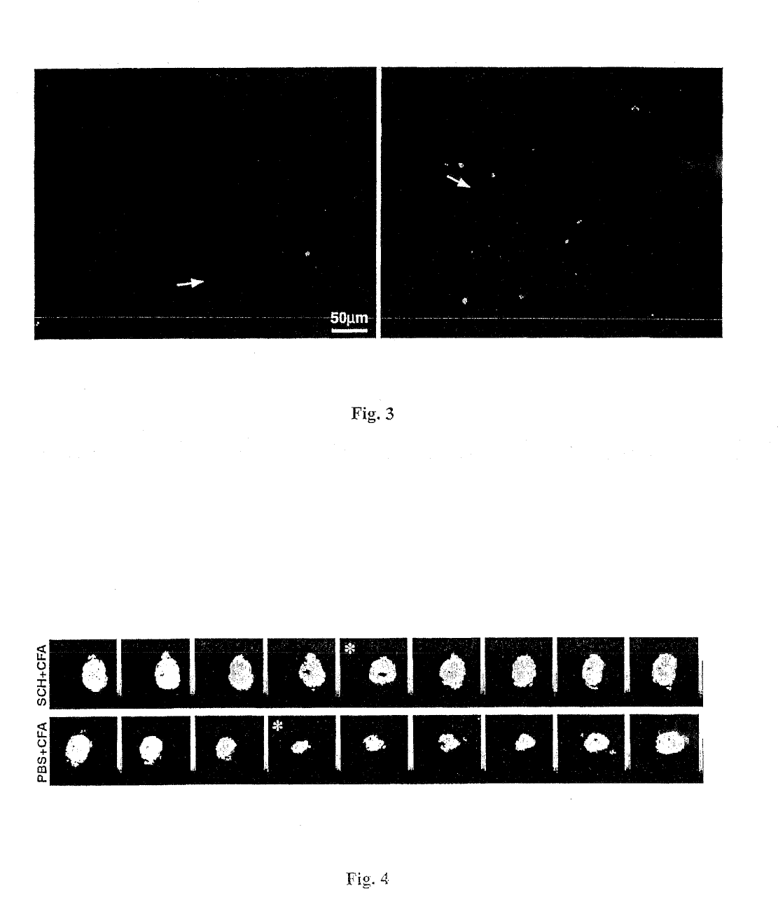

The behavioral results described above were correlated with results obtained by retrograde labeling of rubrospinal neurons in the red nucleus of the brain, following administration of the neurotracer dye Fluoro-ruby below the site of spinal cord contusion. Sections from red nuclei of rats that were immunized with spinal cord homogenate in CFA (0.5 mg/ml) or injected with PBS in the same adjuvant are shown in FIG. 3. As previously reported (Hauben et al., 2000a), the numbers of stained rubrospinal neurons correlated well with the behavioral outcome as measured by the BBB score.

In the diffusion-anisotropy MRI analysis, anisotropy maps of axial slices taken from the spinal cords of rats immunized with spinal cord homogenate in CFA (0.5 mg/ml) showed areas of diffusion anisotropy along the entire length of the cord, and all cords manifested a continuous longitudinal structure (FIG. 4). In contrast, slices taken from the PBS-injected controls showed a loss of organized structure at the center of the lesion site, and the area of diffusion anisotropy in most of the analyzed slices was relatively small (FIG. 4). For rats immunized with spinal cord homogenate, the average sum of anisotropy (SAI)— representing the anisotropy value of the site of the injury (the slice with lowest SAI)— was almost 2-fold higher than the SAI of the same slice taken from the PBS-injected control (290 compared to 167 arbitrary units). The behavioral outcome correlated well with the MRI results: the higher the behavioral score, the larger the area of diffusion anisotropy found at the site of the lesion.

### Example 4

### Active Immunization Promotes Functional Recovery in EAE-Resistant SPD Rats

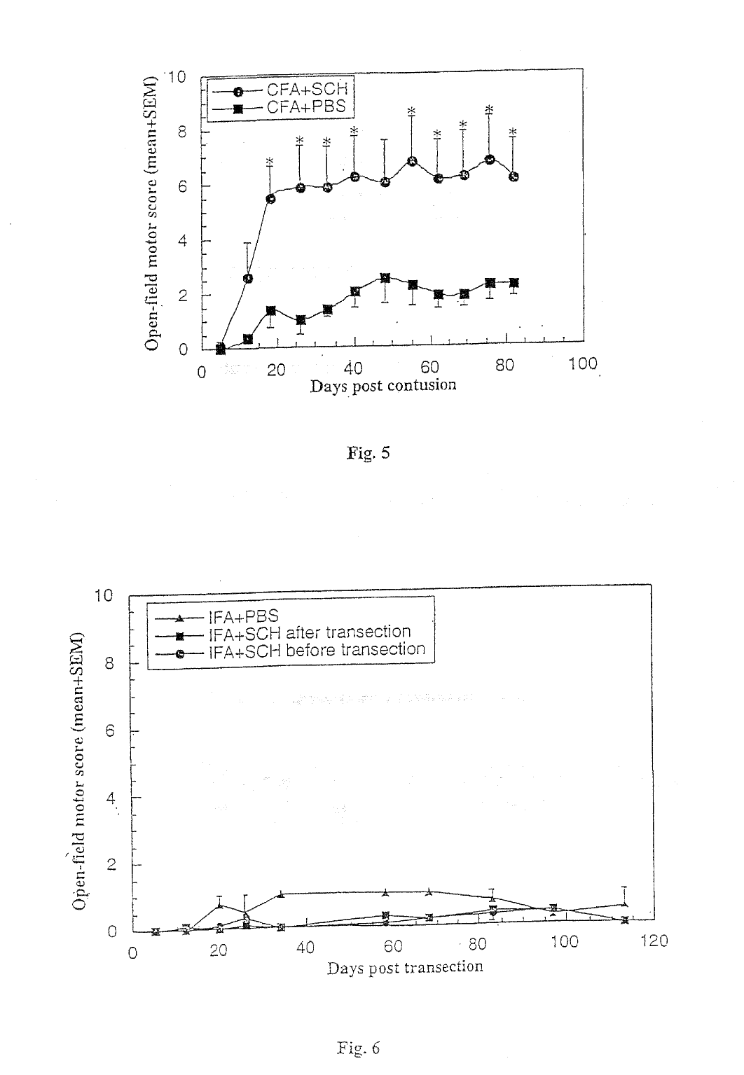

We have recently observed that in Sprague-Dawley (SPD) rats, known to be resistant to induction of EAE, spinal cord contusion evokes an endogenous beneficial immune response. Since no such response could be demonstrated in the EAE-susceptible Lewis rats, it was suggested that this autoimmune neuroprotective response exists in EAE-resistant strains, but not in strains that are susceptible to EAE. It was therefore of interest to determine whether active immunization can further improve the functional recovery in an EAE-resistant strain, possibly by boosting the spontaneous beneficial response. Twelve days before spinal cord contusion, 5 male SPD rats were immunized with spinal cord homogenate emulsified in CFA (containing 0.5 mg/ml bacteria) and 5 were injected with PBS in the same adjuvant (FIG. 5). The immunized rats showed significantly better functional recovery than the PBS-injected controls, starting from day 12 after contusion and at all indicated time points (p<0.05, 2-way ANOVA with replications). This finding, together with our previous observation that functional outcome after spinal cord injury even in EAE-resistant SPD rats can be improved by passive transfer of MBP-reactive splenocytes, suggests that immunization with myelin-associated self-antigens can lead to improved recovery after spinal cord injury in both EAE-resistant and EAE-susceptible rats, presumably by preventing the spread of neural loss.

### Example 5

### Active Immunization with Spinal Cord Homogenate is Ineffective after Complete Spinal Cord Transection

Our previous studies have suggested that immune neuroprotection can prevent complete paralysis after a partial injury to the spinal cord. It was therefore of interest to determine whether active immunization following spinal cord injury is effective in the case of a complete cut. Seven days before complete spinal cord transection, 5 female Lewis rats were immunized with spinal cord homogenate emulsified in IFA and 5 were injected with PBS in IFA. Five more were immunized immediately after the transection. None of these rats showed any significant locomotor function when examined weekly up to 4 months after transection (FIG. 6). No differences could be detected between the 3 experimental groups, suggesting that—as in the case of passive immunization with MBP-reactive T cells (Hauben et al., 2000a)—active immunization with spinal cord homogenate has a neuroprotective effect on axons that survived the direct injury but does not lead to regeneration of completely transected axons.

### Example 6

### Post-Injury Immunization with Altered Peptide Ligands: Neuroprotection with Reduced Risk of Disease.

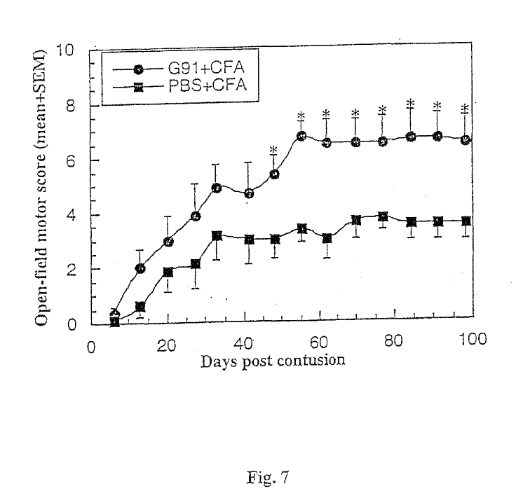

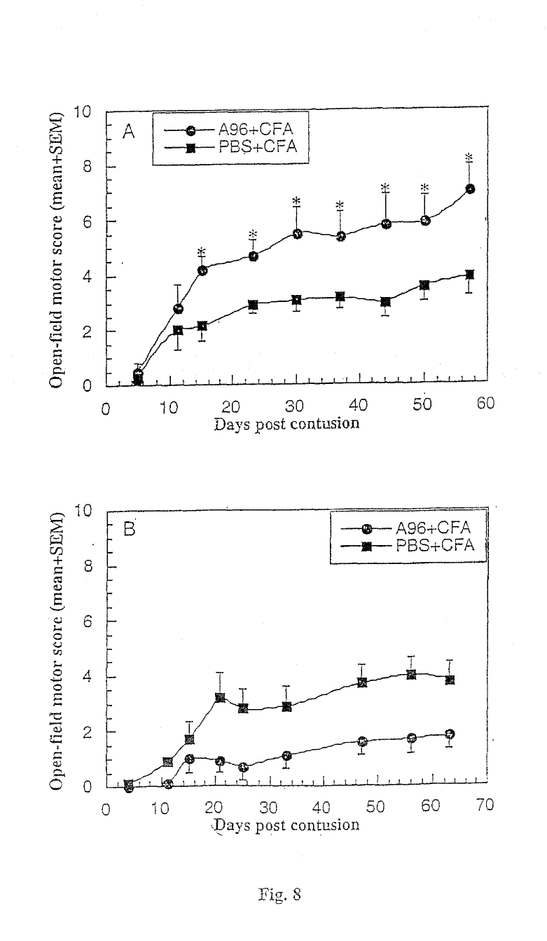

Having established that complete paralysis after spinal cord injury can be prevented by post-injury active immunization using myelin-associated antigens, we then proceeded to search for a way to immunize with a safe (i.e., non-pathogenic) peptide. One way to develop a means of immunization that will benefit the injured spinal cord and will be safe in both susceptible and resistant strains might be to use an altered peptide ligand, for example an encephalitogenic self-peptide modified at its T-cell receptor-binding site in such a way that it will still be presented by the antigen-presenting cell but will no longer be pathogenic (Gaur et al, 1997; Vergelli et al., 1996). A potential approach employs a peptide in which a critical amino acid in the TCR-binding site is modified. In an attempt to produce a suitable antigen, we used the encephalitogenic MBP peptide (amino acids 87-99) in which residue 91 (lysine) was replaced with glycine (G91) or with alanine (A91) or residue 96 (proline) was replaced with alanine (A96). Immunization, immediately after the injury, with either of these apparently non-pathogenic altered peptide ligands emulsified in CFA (0.5 mg/ml bacteria) led to the best recovery of motor activity seen in both Lewis and SPD rats in this study. Directly after severe spinal cord contusion, 5 female Lewis rats were immunized subcutaneously with the altered peptide ligand G91 (100 μg/rat) in CFA (0.5 mg/ml bacteria) and 5 littermates (control) were injected with PBS in the same adjuvant (FIG. 7). Significantly better locomotor function was observed in the G91-immunized rats than in the controls, starting from day 48 after contusion and at all time points of measurement thereafter (p<0.05, 2-way ANOVA with replications). Three uninjured littermates immunized with G91 in CFA showed no EAE symptoms. In a second set of experiments, male SPD rats (n=6 in each group) were immunized, directly after spinal cord contusion, with A96 (100 μg/rat or 500 μg/rat) in CFA (0.5 mg/ml bacteria). Rats immunized with 100 μg A96 performed significantly better than PBS-injected controls, starting from day 15 after contusion and at all time points thereafter (p<0.05, 2-way ANOVA with replications; FIG. 8A). Rats immunized with 500 μg A96 rather than 100 μg, however, performed significantly worse than PBS-injected controls (p<0.05, 2-way ANOVA with replications; FIG. 8B).

### Example 7

### Methylprednisolone (MP) Obliterates the Neuroprotective Effect Induced by A91 Immunization in Both EAE-Susceptible and EAE-Resistant Rats

As shown above, post-traumatic vaccination with A91 improves motor recovery after ISCI in both EAE-susceptible and EAE-resistant rats. Also, administration of MP in high doses does not prevent the development of EAE after active immunization in EAE-susceptible strains (Steiner et al., 1991). In light of these findings, we first combined A91 immunization with high-dose MP, and examined their joint effect on the motor recovery of EAE-susceptible rats subjected to ISCI. Since protective autoimmunity appears to be genetically regulated (Kipnis et al., 2001), we repeated the experiment using EAE-resistant rats. A controlled contusive injury was inflicted on the spinal cords of anesthetized female Lewis (susceptible) or SPD (resistant) rats. After injury, one group of rats from each strain was injected with MP (MP), a second group was immunized with A91, a third group received both MP injection and A91 immunization (A91+MP), and the control group was immunized with PBS in CFA (CFA). Each group contained five rats. MP was administered as a single dose of 30 mg/kg, 10 min after injury, a protocol reported to significantly reduce lesion volumes in rats with injured spinal cords (Yoon et al., 1999). Immunization was given within 1 h after contusion.

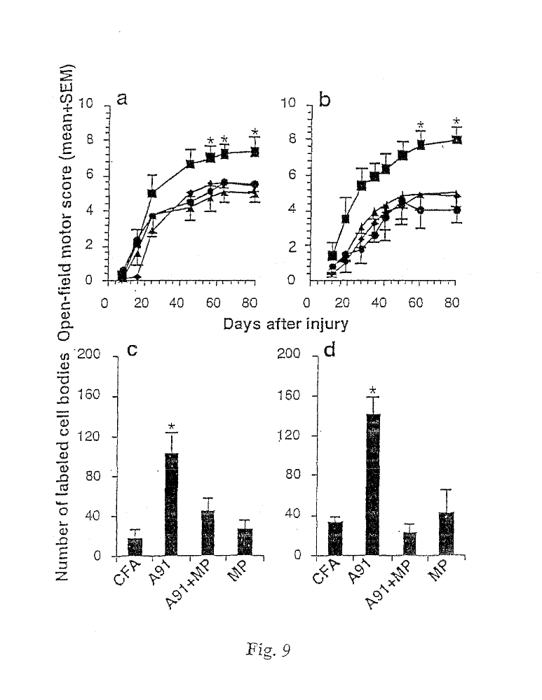

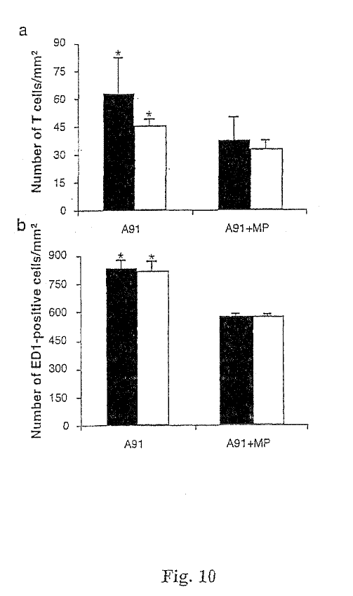

Immunization with A91 alone, as expected, resulted in significantly better motor recovery than that obtained in CFA-treated controls. Administration of MP alone had no significant effect on motor recovery. The highest behavioral scores (mean ±s.e.m.) obtained on the BBB scale (see Methods) by the A91-vaccinated groups were 7.3±0.5 (Lewis rats) and 8.0±0.7 (SPD rats) compared to 5.6±0.3 and 4.5±1, respectively, in the MP-treated rats (P<0.05 for both Lewis and SPD rats; two-tailed t-test) and 5±0.5 or 5±0.7, respectively, in the CFA-treated rats (P<0.05 for both Lewis and SPD rats; two-tailed t-test). However, when treatment with A91 was combined with MP, the beneficial effect of the vaccination was abolished in both Lewis and SPD rats (FIGS. 9a and b). In this case the BBB scores were 5.4±0.3 and 4.9±0.9 for Lewis and SPD rats, respectively (P<0.05; two-tailed t-test, in both, when compared to A91 alone). These behavioral results were correlated with morphological findings obtained by retrograde labeling of rubrospinal neurons in the red nucleus of the brain. The morphological results confirmed that the protective effect of immunization with A91 on neuronal survival is indeed abolished by MP: the numbers of surviving red nucleus cells in the A91-treated rats were 104±19.9 and 143±17 (Lewis and SPD, respectively) compared to 45±12 and 23±8, respectively, after treatment with A91+MP (P<0.05 and <0.004, respectively; two-tailed t-test). These results also show that MP by itself failed to protect red nucleus neurons from secondary degeneration: the numbers of surviving cells in MP-treated rats (28±9 and 43±23 for Lewis and SPD rats, respectively; P<0.05 when compared to A91 alone; two-tailed t-test) were similar to those found in CFA-treated control rats, and significantly lower than those found in A91-treated rats (FIGS. 9c and d).

Since MP is an anti-inflammatory agent, elimination of the vaccination-induced recovery was not surprising as it probably reflects an MP-induced inhibition of the immune response to A91. However, previous findings indicated not only that MP does not inhibit the development of actively-induced EAE, but that the disease can even be exacerbated if MP is administered before or during disease induction (Steiner et al., 1991). These findings prompted us to examine whether MP indeed inhibits the immune response to A91. Female Lewis and SPD rats were subjected to severe spinal cord contusion and then treated with CFA, A91, AP91+MP, or MP alone (n=3 in each group). Lymph node cells, isolated 10 days after injury, were cultured in the presence of A91, MBP, OVA, Con A, or no antigen. Measurement of the proliferative response to these antigens showed that MP prevents the specific proliferative response to A91 in both Lewis and SPD rats treated concomitantly with A91 vaccination and MP. In Lewis rats, the stimulation index (SI, see methods), calculated as proliferation in the presence of A91 relative to proliferation in an antigen free medium, for treatment with A91+MP was 1.3 whereas for treatment with A91 alone it was 2.1 (P<0.05, two-tailed t-test) (Table 2). In SPD rats, the SI for treatment with A91+MP was also significantly lower (1.2) than that obtained for treatment with A91 (2.8; P<0.05, two-tailed t-test) (Table 2). These results confirmed that the effect of MP on the A91-induced beneficial autoimmunity results from inhibition of the immune response evoked by the vaccination.

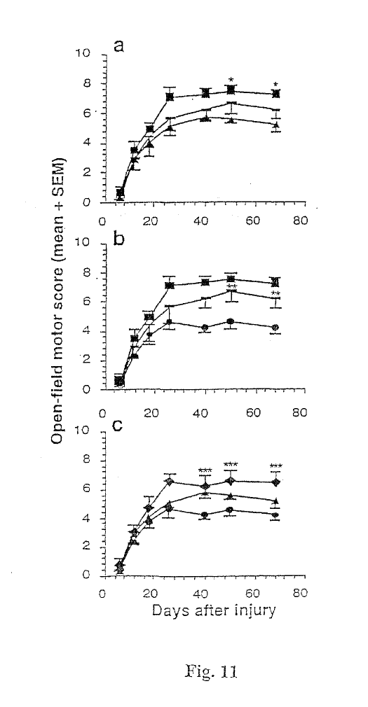

To determine whether MP also affects the availability of T cells and macrophage/macroglia at the site of injury, we subjected Lewis and SPD rats to severe ISCI and treated them immediately after the injury with A91 alone or with A91+MP. Morphological analysis of spinal cords excised 7 days later showed that the inflammatory reaction induced by A91 in both strains was significantly diminished by MP. After treatment with A91+MP, the numbers of T cells per square millimeter recruited at the injury site were 38±3 and 33±3 for Lewis and SPD rats, respectively, compared to 63±12 and 46±5, respectively, after treatment with A91 alone (P<0.05; two-tailed t-test). Treatment with MP in addition to the vaccination also significantly reduced the numbers of ED1-positive cells at the site of injury (834±11 and 817±9 for A91-treated Lewis and SPD rats, respectively, compared to 578±54 and 580±25, respectively, after treatment with A91+MP) (P<0.05 for Lewis rats and P<0.001 for SPD rats; two-tailed t-test) (FIG. 10).

### Example 8

### Injection of Methylprednisolone Immediately after ISCI does not Prevent Promotion of Recovery by Delayed Vaccination with A91

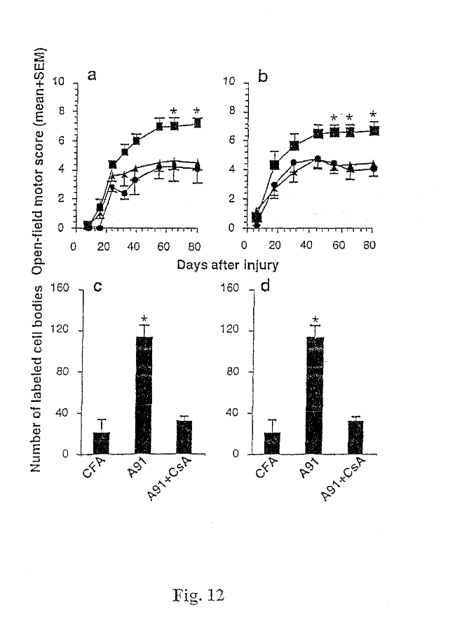

The above findings showed that a single dose of MP (30 mg/kg) had no significant effect by itself on motor recovery, and that in addition, as expected from the known anti-inflammatory activity of this drug, it inhibited the immune response evoked by concurrently administered A91. In an attempt to obtain at least some beneficial effect of MP on motor recovery when given by itself, and to avoid its interference with the effects of the vaccination, we repeated the above experiment giving more doses of MP and delaying the therapeutic vaccination. SPD rats were subjected to severe ISCI and were then divided into five therapeutic groups (n=6 in each group): (a) PBS immediately after injury and CFA-PBS 48 h later; (b) PBS immediately and CFA-A91 48 h later; (c) MP immediately and CFA-A91 48 h later; (d) MP by itself, immediately after injury; and (e) CFA-A91 by itself, immediately after injury. In all cases, treatment with MP (30 mg/kg) or PBS (0.1 ml) was administered three times (5 min, 2 h, and 4 h after injury). The results show that despite the increased number of doses, MP administration did not improve the motor recovery of injured rats (the highest BBB motor score attained was 4.7±0.6, mean ±s.e.m.; FIG. 11). Moreover, starting 40 days after injury, MP-treated rats obtained the lowest open-field motor score of all the groups (4.3±0.3 compared to 5.8±0.4 for PBS-treated rats). The highest motor score (7.5±0.7) was obtained by rats immunized with A91 immediately after the injury. Up to the end of the study, even rats that were immunized 48 h after injury showed better motor recovery (6.4±0.8) than rats treated with CFA alone (5.2±0.5) or MP alone (4.2±0.4). Interestingly, delaying the immunization by 48 h relative to MP treatment appeared to circumvent the inhibitory effect of MP: the motor recovery score of rats treated with both MP and delayed immunization (6.5±0.7) was very similar to that of rats treated with delayed immunization only (6.4±0.8). These results strongly suggest that MP by itself, even in increased amounts, is not beneficial in the model of ISCI. The data further indicate that MP does not interfere with the effects of a therapeutic vaccination administered 48 h after injury.

### Example 9

### Neuroprotection Induced by Immunization with A91 is Inhibited by Cyclosporin-A in Both EAE-Susceptible and EAE-Resistant Rats

MP-induced inhibition of motor recovery was found here to be correlated with inhibition of the proliferative response to A91 (see Tables 2 and 3). Cyclosporin-A (CsA) is an anti-inflammatory agent that inhibits the function of T cells. In addition, CsA has been successfully used to treat EAE. Interestingly, however, EAE development is not inhibited by low doses of this drug. It is possible that the dialog between T cells and APCs at the site of injury, an essential prerequisite for neuroprotection, might be inhibited by the CsA.

To examine this possibility we treated rats, immediately after a severe contusion, with CFA, A91 alone, or A91+CsA. In both Lewis and SPD rats, the protective effect induced by immunization with A91 was abolished by treatment with CsA, motor recovery after treatment with A91+CsA (BBB scores of 4.1±1 for Lewis rats and 4.1±0.5 for SPD rats) was significantly poorer than that observed after immunization with A91 only (7.2±0.4 for Lewis rats and 6.7±0.6 for SPD rats; P<0.05 in both cases, two-tailed t-test) (FIGS. 12a and b). The results were supported by morphological examination of neuronal survival in the red nuclei of these rats (FIGS. 12c and d).

### Discussion

The results above show that immunization of animals with a myelin-derived modified peptide can be beneficial in cases of severe spinal cord contusion even if performed immediately after, rather than before, the injury. The benefit is manifested by an improved recovery of motor performance, and can be achieved in rat strains that are prone to autoimmune disease development as well as in those that are not. The observed differences between the strains in their rate of recovery from spinal cord injury appears to be attributable to the differences in their ability to sustain a T-cell-dependent beneficial autoimmune response.

The findings according to the present invention support our contention that beneficial autoimmunity is a physiological response to trauma, and that it is amenable to boosting. It appears that EAE-susceptible strains lack the control mechanism which promotes the physiological beneficial autoimmune response, but that this response can nevertheless be induced by immunization, depending on the choice of antigen and adjuvant. In resistant strains the spontaneous immune response to trauma might be restricted.

In searching among the CNS injury-associated proteins for a “safe” (non-pathogenic) antigen to boost the endogenous response we examined the post-traumatic effect of a peptide which, though originally encephalitogenic, was modified by the replacement of a single amino acid in its TCR-binding site, a manipulation which attenuated the pathogenic effect. The peptide used was the amino acid sequence 87-99 of MBP, with glycine or alanine substituting for lysine in position 91 (G91 and A91, respectively) or alanine substituting for proline in position 96 (A96). These peptides were shown not to cause EAE in susceptible strains even when injected after emulsification in a potent adjuvant such as CFA (Gaur et al., 1997; and Vergelli et al., 1996). We found that these peptides, when emulsified in CFA, exert a neuroprotective effect following a single vaccination given immediately after spinal cord contusion. It thus seems that it is possible to design a beneficial autoimmunity which, even in susceptible strains, is not accompanied by the risk of autoimmune disease. Recent studies have warned that the therapeutic use of such modified peptides for patients with multiple sclerosis may not be safe as these peptides may, when administered at high dosage to patients predisposed to pathogenic autoimmunity, aggravate the disease as a result of their cross-reactivity with MBP (Bielekova et al., 2000; and Kappos et al., 2000). It should however be emphasized that in individuals with an autoimmune disease the treatment of choice is immune suppression, as immune deviation (though possibly beneficial) is a less safe option. Immune neuroprotection requires an active immune response and therefore can exert a therapeutic effect through immune boosting with encephalitogenic or non-encephalitogenic peptides using self- or altered self-peptides, but not through immune suppression. We found that vaccination of rats with a modified MBP peptide at a dosage high enough to cause immune suppression indeed had no neuroprotective effect or even had a negative effect due to loss of the endogenous beneficial response.

The results according to the invention show that in rats with spinal contusion, complete paralysis can be prevented and recovery thus promoted by post-traumatic immunization with the MBP-derived synthetic analogue peptides. They also showed that both susceptible and resistant strains can benefit from post-injury immunization, and that the beneficial effect on neuroprotection is influenced by the choice of both the peptide and the adjuvant.

Active vaccination with non-encephalitogenic peptides has produced promising results in rats with ISCI. We considered the possibility that the neurological outcome after ISCI might be further improved if we combined the vaccination approach with treatment that provides immediate protection of neural tissue. We therefore examined the effects of combining protective autoimmune vaccination with methylprednisolone (MP), currently the only clinically approved therapy for ISCI.

MP has been shown to improve neurological recovery after insult to the spinal cord. The observed anti-oxidant and anti-inflammatory effects of MP have made it the drug of choice for preventing some of the destructive events triggered immediately after injury. Since it is not unrealistic to expect that therapeutic vaccination might become accepted clinical practice for the treatment of ISCI, it was of interest to examine the effect of a combination of MP treatment with A91 vaccination. We thought it possible, however, that the anti-inflammatory effect of MP might interfere with the beneficial effect of active vaccination (which evokes inflammation). To examine this possibility, we first administered a high dose of MP at the same time as A91 immunization. As expected, MP inhibited the beneficial effect of A91 vaccination on the functional outcome. This inhibition was correlated with a significant reduction, in both EAE-susceptible and EAE-resistant rats, in the immune response to A91, as well as in the accumulation of immune cells in the spinal cords of the vaccinated animals. The beneficial effect of the therapeutic vaccination was similarly abolished by CsA.

On the other hand, when vaccination with A91 was delayed for 48 h after injury, there was no interference with its effect by the anti-inflammatory action of the MP injected immediately after the injury. This finding suggests that, whatever the mechanism of MP action, its effect is transient and does not interfere with later treatment even if that treatment is immune-related. Thus, these findings convey two important messages: that vaccination with A91 is neuroprotective even if administered 48 h after injury; and that MP, even when administered in several high doses, does not confer neuroprotection.

In the present study, the number of recruited immune cells at the site of injury was significantly reduced in both EAE-susceptible and EAE-resistant rats treated with a combination of MP and A91. It is shown herein that inhibition of this response by anti-inflammatory agents results in loss of the functional benefit induced by therapeutic vaccination. These data support our current suggestion that the accumulation and activation of T cells, as well as their interaction with other immune cells at the site of injury, are important elements in protective autoimmunity. In addition, and contrary to common opinion, this work provides further evidence for the important role of inflammation in functional recovery after spinal cord injury, and argues in favor of modulating the inflammatory response rather than preventing it.

Owing to the widespread clinical use of MP, inflammation after spinal cord injury has been considered to have only a harmful effect; however, this widely prevalent view is still controversial. Studies have provided persuasive evidence for the key role of the immune system in protecting neural tissues and inducing regrowth after injury (Rapalino et al., 1998). In addition, even when inflammatory cells are efficiently removed from the site of injury, the volume of early tissue loss is not decreased (Bartholdi and Schwab, 1995). These findings suggest that the use of anti-inflammatory agents after ISCI should be carefully re-evaluated.

The use of MP for treatment of ISCI has become a controversial issue, as several other recent works have also raised questions about its usefulness. Furthermore, in some cases MP appears to be more harmful than beneficial. As shown above, treatment with MP, either as a single high dose or as a few incremental doses, failed to improve motor recovery assessed morphologically and functionally. Rats treated with MP only (in several doses) obtained a lower BBB locomotor score than that of control rats (although the difference was not significant). Since high-dose MP therapy is associated with serious side effects, and because its beneficial effect on neurological recovery has not been unequivocally confirmed, it seems that the time has come to reconsider whether the supposed benefits of this therapy outweigh its potential risks.

Therapeutic vaccination with non-encephalitogenic peptides appears to be a promising treatment for ISCI in humans; however, even if MP continues to be routinely prescribed as well, it appears that the beneficial effect of the vaccination need not be neutralized. As demonstrated in this study, therapeutic vaccination, even when given as late as 48 h after injury, is still effective in improving the motor outcome. This time window has the advantage of allowing the therapeutic effect of the delayed vaccination to be exerted even if MP is given immediately after the injury. This finding suggests that the changes induced by MP are reversible, at least in part, for up to 48 h after its administration.

In summary, our study indicates that anti-inflammatory agents administered immediately after injury, if given concomitantly with therapeutic vaccination, abolish the benefit of the vaccination. We also demonstrated the failure of MP to promote neuroprotection. It should be noted, however, that the apparent lack of effect of MP on functional activity and neuronal survival is not incompatible with an immediate, transient, beneficial effect of MP that is outweighed by the need for immune cells. This suggestion is based on recent observations by our group indicating that the mechanism of well-controlled protective immunity includes a stage where the tissue pays a price, in terms of neuronal loss, for the overall benefit (U. Nevo et al., unpublished observations). These results, taken together, strongly suggest that specific modulation of the immune response, rather than its general suppression, should be considered as a therapy for ISCI.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

TABLE 1
Active immunization with myelin basic protein 7 days before
or immediately after spinal contusion promotes recovery.

| | | Maximal BBB | Two- |
|---|---|---|---|
| Time of | | score | tailed |
| immunization | Treatment | (Mean ± SE) | t-test |
| 7 days | MBP in IFA | 6.1 ± 0.7 | p < 0.02 |
| before | | (n = 5) | |
| injury | PBS in IFA | 3.0 ± 0.7 | |
| | | (n = 5) | |
| Immediately | MBP in IFA | 6.4 ± 0.9 | |
| after | | (n = 5) | |
| injury | PBS in IFA | 4.3 ± 0.9 | p = 0.18 |
| | | (n = 5) | |

Female Lewis rats were immunized with MBP in IFA or injected with PBS in IFA. Immediately before immunization or 7 days after it the rats were deeply anesthetized, laminectomized, and subjected to spinal cord contusion at T8. Locomotor behavior in an open field was scored. Maximal values and statistical differences revealed by a 2-tailed Student's t-test are presented. Both vaccination protocols led to improved motor performance relative to the PBS-treated controls, though the neuroprotective autoimmune response was boosted more effectively by the vaccination administered 7 days prior to the injury.

TABLE 2
Proliferating lymph node cells of injured Lewis rats

| Groups | No antigen | MBP | OVA | A91 | ConA |
|---|---|---|---|---|---|
| CFA + PBS | 406 ± 14^{a} | 507 ± 35 | 520 ± 28 | 534 ± 54 | 20316 ± 1676 |
| CFA + A91 | 1456 ± 320 | 1534 ± 60 | 1459 ± 272 | 3196 ± 586* | 23064 ± 1279 |
| A91 + MP | 506 ± 20 | 673 ± 70 | 637 ± 56 | 680 ± 73 | 20376 ± 1936 |
| MP | 352 ± 60 | 394 ± 64 | 278 ± 46 | 373 ± 52 | 12042 ± 1327 |

^{a}mean ± SEM;
*P = 0.001, Student's t-test, A91 versus A91 + MP.

TABLE 3
Proliferating lymph node cells of injured SPD rats

| Groups | No antigen | MBP | OVA | A91 | ConA |
|---|---|---|---|---|---|
| CFA + PBS | 325 ± 47^{a} | 360 ± 50 | 467 ± 68 | 411 ± 38 | 28062 ± 2291 |
| CFA + A91 | 519 ± 47 | 695 ± 129 | 664 ± 78 | 1488 ± 255* | 9792 ± 2958 |
| A91 + MP | 309 ± 55 | 419 ± 71 | 430 ± 77 | 393 ± 61 | 6660 ± 849 |
| MP | 508 ± 98 | 586 ± 47 | 655 ± 77 | 611 ± 50 | 23778 ± 5081 |

^{a}mean ± SEM;
*P = 0.001, Student's t-test, A91 versus A91 + MP.

### REFERENCES

Bartholdi, D. & Schwab, M. E. Methylprednisolone inhibits early inflammatory processes but not ischemic cell death after experimental spinal cord lesion in the rat. Brain Res. 672, 177-186 (1995).
Basso, D. M., Beattie, M. S. and Bresnahan, J. C. “A sensitive and reliable locomotor rating scale for open field testing in rats”, J. Neurotrauma 12, 1-21 (1995).
Basso, D. M., Beattie, M. S. & Bresnahan, J. C. “Graded histological and locomotor outcomes after spinal cord contusion using the NYU weight-drop device versus transection. Exp. Neurol. 139, 244-256 (1996).
Bazan, N. G, Rodriguez de Turco, E. B and Allan, G. “Mediators of injury in neurotrauma: intracellular signal transduction and gene expression”, J. Neurotrauma 12, 791-814 (1995)
Behrmann, D. L., Bresnahan, J. C. & Beattie, M. S. “Modeling of acute spinal cord injury in the rat: neuroprotection and enhanced recovery with methylprednisolone, U-74006F and YM-14673”, Exp. Neurol. 126, 61-75 (1994)
Ben-Nun, A. and Cohen, I. R. Experimental autoimmune encephalomyelitis (EAE) mediated by T cell lines: process of selection of lines and characterization of the cells. J. Immunol. 129, 303-308 (1982).
Bielekova, B., et al. Encephalitogenic potential of the myelin basic protein peptide (amino acids 83-99) in multiple sclerosis: Results of a phase II clinical trial with an altered peptide ligand. Nat. Med. 6, 1167-1175 (2000).
Brewer, K. L., Bethea, J. R. & Yezierski, R. P. Neuroprotective effects of interleukin-10 following excitotoxic spinal cord injury. Exp. Neurol. 159, 484-493 (1999).
Burns, J., Rosenzweig, A., Zweiman, B. & Lisak, R. P. Isolation of myelin basic protein-reactive T-cell lines from normal human blood. Cell Immunol. 81, 435-440 (1983).
Butovsky, O., Hauben, E. & Schwartz M. Morphological aspects of spinal cord autoimmune neuroprotection: colocalization of T cells with B7-2 (CD86) and prevention of cyst formation. FASEB J. 25, 1065-1067 (2001).
Constantini, S. & Young, W. The effects of methylprednisolone and the ganglioside GM1 on acute spinal cord injury in rats. J. Neurosurg. 80, 97-111 (1994).
Crowe, M. J., Bresnahan, J. C., Shuman, S. L., Masters, J. N. & Beattie, M. S. Apoptosis and delayed degeneration after spinal cord injury in rats and monkeys [published erratum appears in Nat. Med. 3, 240]. Nat. Med. 3, 73-76 (1997).
Faden, A. I. Experimental neurobiology of central nervous system trauma. Crit. Rev. Neurobiol. 7, 175-186 (1993).
Gaur, A, et al. Amelioration of relapsing experimental autoimmune encephalomyelitis with altered myelin basic protein peptides involves different cellular mechanisms. J. Neuroimmunol. 74, 149-158 (1997).
Hammarberg, H., et al. Neuroprotection by encephalomyelitis: rescue of mechanically injured neurons and neurotrophin production by CNS-infiltrating T and natural killer cells. J. Neurosci. 20, 5283-5291 (2000).
Hauben, E., et al. Passive or active immunization with myelin basic protein promotes recovery from spinal cord contusion. J. Neurosci. 20, 6421-6430 (2000a).
Hauben, E., et al. Autoimmune T cells as potential neuroprotective therapy for spinal cord injury [letter]. Lancet 355, 286-287 (2000b).
Hirschberg, D. L., et al., 1998, J. Neuroimmunol. 89, 88-96
Kamholz et al., Proc. Natl. Acad. Sci. USA, 83, 4962-4966 (1986)
Kappos, L., et al. Induction of a non-encephalitogenic type 2 T helper-cell autoimmune response in multiple sclerosis after administration of an altered peptide ligand in a placebo-controlled, randomized phase II trial. Nat. Med. 6, 1176-1182 (2000).
Karin et al., 1998, J. Neuroimmunol. 160:5188-5194
Killen, J. A. & Swanborg, R. H. Autoimmune effector cells. III. Role of adjuvant and accessory cells in the in vitro induction of autoimmune encephalomyelitis. J. Immunol. 129, 759-763 (1982).
Kipnis, J., et al. T cell immunity to copolymer 1 confers neuroprotection on the damaged optic nerve: possible therapy for optic neuropathies. Proc. Natl. Acad. Sci. USA 97, 7446-7451 (2000).
Kipnis, J. et al. Neuronal survival after CNS insult is determined by a genetically encoded autoimmune response. J. Neurosci. 21, 4564-4571 (2001).
Moalem, G., et al. Autoimmune T cells protect neurons from secondary degeneration after central nervous system axotomy. Nat. Med. 5, 49-55 (1999a).
Moalem, G., Monsonego, A., Shani, Y., Cohen, I. R. & Schwartz, M. Differential T cell response in central and peripheral nerve injury: connection with immune privilege. FASEB J. 13, 1207-1217 (1999b).
Moalem, G., et al. Autoimmune T cells retard the loss of function in injured rat optic nerves. J. Neuroimmunol. 106, 189-197 (2000).
Mor and Cohen, J. Immunol. 155: 3693-3699 (1995).
Namikawa, T., Richert, J. R., Driscoll, B. F., Kies, M. W. & Alvord, E. C. Jr. Transfer of allergic encephalomyelitis with spleen cells from donors sensitized with myelin basic protein in incomplete Freund's adjuvant. J. Immunol. 128, 932-934 (1982).
Ota, K. et al., 1990 Nature 346:183-187
Pette, M., et al. Myelin autoreactivity in multiple sclerosis: recognition of myelin basic protein in the context of HLA-DR2 products by T lymphocytes of multiple-sclerosis patients and healthy donors. Proc. Natl. Acad. Sci. USA 87, 7968-7972 (1990).
Popovich, P. G., Whitacre, C. C. & Stokes, B. T. Is spinal cord injury an autoimmune disease? The Neuroscientist 4, 71-76 (1998).
Popovich, P. G., Stokes, B. T. & Whitacre, C. C. Concept of autoimmunity following spinal cord injury: possible roles for T lymphocytes in the traumatized central nervous system. J. Neurosci. Res. 45, 349-363 (1996).
Rapalino, O., et al. Implantation of stimulated homologous macrophages results in partial recovery of paraplegic rats. Nat. Med. 4, 814-821 (1998).
Schluesener, H. J. & Wekerle, H. Autoaggressive T lymphocyte lines recognizing the encephalitogenic region of myelin basic protein: in vitro selection from unprimed rat T lymphocyte populations. J. Immunol. 135, 3128-3133 (1985).
Schwartz, M. & Cohen, I. R. Autoimmunity can benefit self-maintenance. Immunol. Today 21, 265-268 (2000).
Schwartz, M., Moalem, G., Leibowitz-Amit, R. & Cohen, I. R. Innate and adaptive immune responses can be beneficial for CNS repair. Trends Neurosci. 22, 295-299 (1999).
Smolen, A. J., Wright, L. L. & Cunningham, T. J. Neuron numbers in the superior cervical sympathetic ganglion of rat: a critical comparison of methods for cell counting. J. Neurocytol. 12, 739-750 (1983).
Steiner, I., Brenner, T., Mizrachi-Kol, R. & Abramsky, O. Development of experimental allergic encephalomyelitis during steroid administration. Outcome of neurological immune-mediated disorders under immunosuppressive therapy. Isr. J. Med. Sci. 27, 365-368 (1991).
Vergelli, M., et al. Differential activation of human autoreactive T cell clones by altered peptide ligands derived from myelin basic protein peptide (87-99). Eur. J. Immunol. 26, 2624-2634 (1996).
Yoon, D. H., Kim, Y. S. & Young, W. Therapeutic time window for methylprednisolone in spinal cord injured rat. Yonsei Med. J. 40, 313-320 (1999).

1. A method for reducing or inhibiting secondary neuronal degeneration in the central nervous system (CNS) or peripheral nervous system (PNS), comprising
(a) administering to an individual in need thereof an effective amount of an agent selected from the group consisting of a peptide obtained by modification of a self-peptide derived from a CNS-specific antigen, which modification consists in the replacement of one or more amino acid residues of the self-peptide by different amino acid residues, said modified CNS peptide still being capable of recognizing the T-cell receptor recognized by the self-peptide but with less affinity (hereinafter “modified CNS peptide”), wherein said modified CNS peptide is administered in such a manner as to activate T cells in vivo, thereby producing a population of T cells that accumulate at a site of injury, disease, disorder or condition of the CNS or PNS; or
(b) administering an effective amount of T cells that are activated against said modified CNS peptide, said T cells accumulating at a site of injury, disease, disorder or condition of the CNS or PNS;
thereby reducing secondary neuronal degeneration at that site and thus ameliorating the effects of said injury, disease, disorder or condition in the CNS or PNS.

2. The method of claim 1, wherein the individual in need is one suffering from a secondary degeneration which follows a primary CNS injury.

3. The method of claim 2 in which the injury is spinal cord injury, blunt trauma, penetrating trauma, hemorrhagic stroke, or ischemic stroke.

4. The method of claim 3, wherein the injury is spinal cord injury.

5. The method of claim 1, wherein the individual in need is one suffering from a disease, disorder or condition that results in a degenerative process.

6. The method of claim 5 in which the disease, disorder or condition is diabetic neuropathy, senile dementia, Alzheimer's disease, Parkinson's Disease, facial nerve (Bell's) palsy, glaucoma, Huntington's chorea, amyotrophic lateral sclerosis, non-arteritic optic neuropathy, or a peripheral neuropathy.

7. The method of claim 1, wherein said CNS-specific antigen defined in (a) is selected from the group consisting of myelin basic protein (MBP), myelin oligodendrocyte glycoprotein (MOG), proteolipid protein (PLP), myelin-associated glycoprotein (MAG), S-100, β-amyloid, Thy-1, P0, P2, and neurotransmitter receptors.

8. The method of claim 7, wherein said CNS-specific antigen is MBP (SEQ ID NO:1).

9. The method of claim 8, wherein said modified CNS peptide is obtained by modification of a self-peptide selected from the group consisting of p11, p51-70, p87-99, p91-110, p131-150, and p151-170 of MBP.

10. The method of claim 9, wherein said modified CNS peptide is obtained by modification of the self-peptide p87-99 of MBP.

11. The method of claim 10, wherein said modified CNS peptide is obtained by replacing the lysine residue 91 of the peptide p87-99 of MBP with glycine (G91).

12. The method of claim 10, wherein said modified CNS peptide is obtained by replacing the lysine residue 91 of the peptide p87-99 of MBP with alanine (A91).

13. The method of claim 10, wherein said modified CNS peptide is obtained by replacing the proline residue 96 of the peptide p87-99 of MBP with alanine (A96).

14. The method of claim 1, wherein said activated T cells of (c) are selected from the group consisting of autologous T cells, allogeneic T cells from related donors, HLA-matched or partially matched semi-allogeneic donors, and HLA-matched or partially matched fully allogeneic donors.

15. The composition of claim 14, wherein said activated T cells are semi-allogeneic T cells.

16. The method of claim 1, wherein said activated T cells are autologous T cells.

17. The method of claim 16, in which the injury is spinal cord injury, blunt trauma, penetrating trauma, hemorrhagic stroke, or ischemic stroke.

18. The method of claim 17, wherein the injury is spinal cord injury.

19. The method of claim 16, wherein the individual in need is one suffering from a disease, disorder or condition that results in a degenerative process.

20. The method of claim 19, in which the disease, disorder or condition is diabetic neuropathy, senile dementia, Alzheimer's disease, Parkinson's Disease, facial nerve (Bell's) palsy, glaucoma, Huntington's chorea, amyotrophic lateral sclerosis, non-arteritic optic neuropathy, or a peripheral neuropathy.

21. The method of claim 16, wherein said autologous T cells have been stored or are derived from autologous central nervous system cells.

22. The method of claim 1 wherein said agent is administered intravenously, orally, intranasally, intrathecally, intramuscularly, intradermally, topically, subcutaneously, mucosally or buccally.


SEQUENCE LISTING (1 sequence)

SEQ ID NO: 1 - protein, 171 aa, Artificial
  Synthetic
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSTGAEGQRPGFGYGGRASDYKSAHKGFKGVDAQGTLSKIFKLGGRDSRSGSPMARR